(19) United States
(12) Patent Application Publication
(10) Pub. No.: US 2010/0143278 A1
(43) Pub. Date: Jun. 10, 2010

(54) Low-residue deodorant or antiperspirant stick based on an oil-in-water emulsion

(76) Inventors: Bernhard Banowski, Duesseldorf (DE); Armin Wadle, Erkrath (DE); Marcus Claas, Hilden (DE)

Correspondence Address: Henkel Corporation, 10 Finderne Avenue, Bridgewater, NJ, 08807, US

(21) Appl. No.: 12/622,755

(22) Filed: Nov. 20, 2009

Related U.S. Application Data
(63) Continuation of application No. 11/192,389, filed on Jul. 28, 2005.
(60) Provisional application No. 60/600,688, filed on Aug. 11, 2004.

(30) Foreign Application Priority Data
Jul. 28, 2004 (DE) 10 2004 036 689.6

(51) Int. Cl. A61K 8/28; A61Q 15/00; A61K 8/27; A61K 8/26; A61K 8/58

(52) U.S. Cl. 424/66; 424/65; 424/67; 424/68

(57) ABSTRACT

Deodorant or antiperspirant sticks based on an oil-in-water dispersion for application to the skin.

### CROSS-REFERENCE TO RELATED APPLICATIONS.

This Application claims priority under 35 U.S.C. §119 of German Patent Application No. DE 10 2004 036 689.6, filed Jul. 28, 2004, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to a deodorant or antiperspirant stick based on an oil-in-water dispersion for the application of water-soluble active ingredients to the skin.

(2) Description of Related Art, Including Information Disclosed Under 37 C.F.R. §§1.97 and 1.98

Standard commercial deodorants and antiperspirants are mostly formulated as sprays or as sticks; there are also roll-on preparations and creams in the market. Many stick antiperspirant preparations are formulated as anhydrous suspension sticks. Preparations of this type leave behind a pleasant dry feel on the skin for the user following application. However, effective release of the water-soluble antiperspirant active ingredients from such preparations is limited (cf.: Chemistry and Technology of the Cosmetics and Toiletries Industry, editors: D. F. Williams and W. H. Schmitt, London: Blackie, 1996, 2nd edition, p. 326), and in many cases the feeling of freshness valued by many consumers is not achieved. The anhydrous preparations, in particular those based on volatile silicone oils, have the disadvantage that the dispersed active ingredients readily lead to visible product residues on skin and clothing. Furthermore, such preparations are relatively expensive since the oil components are more expensive as active ingredient carriers than water. Compression during application often results in the loss of oil, which reduces the cosmetic acceptance of these preparations for the user.

Compared with anhydrous sticks, as are known, for example, from WO 94/24997 A1 and WO 00/67713 A1, emulsion sticks, as are disclosed, for example, in WO 98/17238 A1, EP 281 288 A2, DE 2 335 549, U.S. Pat. No. 4,725,431, EP 617 952 A1 and EP 291 334, have a number of advantages. Replacing the wax and oil additives with water makes the emulsion sticks more cost-effective to manufacture. The emulsified waxes convey a soft, gentle feel on the skin, and, finally, water-soluble cosmetic active ingredients (i.e., in particular, also antiperspirant active ingredients) can more readily be released onto the skin since they are already present in dissolved form in the aqueous phase of the emulsion.

Since the emulsion sticks of the cited prior art are formulated on the basis of a water-in-oil dispersion, the water-soluble active ingredients are present in the inner, dispersed phase and, following application, must first migrate through the outer, lipophilic layer in order to reach their site of action on the skin. The known water-in-oil emulsion sticks thus have disadvantages which are similar to those of anhydrous suspension sticks with regard to the availability of active ingredient.

DE 19749819 A1 discloses water-containing and oil-containing, wax-free antiperspirant sticks based on an oil-in-water emulsion. Sticks of this type have inadequate cosmetic properties, leave behind unpleasant sticky and visible residues and exhibit a stability which is insufficient for prolonged use. One example with glycerol monostearate as W/O emulsifier and octyldodecanol as oil component has a medium-firm consistency and a greasy feel on the skin and begins to soften at just 50° C.

WO 99/59537 A1 discloses hydrous, oil-free deodorant sticks based on a (lipid or oil)-in-water emulsion which comprises wax components with a melting point of >50° C., nonionic water-in-oil emulsifiers, a nonionic oil-in-water emulsifier with an HLB value of more than 7 and a polyol. In addition, this document discloses microemulsion-containing and PIT emulsion-containing deodorant sticks which comprise wax components with a melting point of >50° C., nonionic water-in-oil emulsifiers, a nonionic oil-in-water emulsifier with an HLB value of more than 7 and oils which are liquid at 20° C. Sticks of this type likewise have inadequate cosmetic properties, leave behind unpleasant sticky and visible residues and exhibit a stability which is inadequate for prolonged use.

WO 02/083091 A1 discloses structured antiperspirant compositions in the form of an oil-in-water microemulsion. The structural difference between these compositions and the oil-in-water dispersion sticks of the present invention becomes particularly clear because of the high fraction of silicone and hydrocarbon oils which are unfavorable according to the invention.

WO 02/017870 A2 discloses hydrous antiperspirant sticks which comprise a siliconized polyamide as consistency regulator or structurant.

WO 02/032914 A1 discloses hydrous antiperspirant sticks which comprise acylated cellobiose as consistency regulator or structurant and a high fraction of silicone and hydrocarbon oils, which are unfavorable according to the invention.

The laid-open specifications DE 199 62 878 A1 and DE 199 62 881 A1 disclose deodorant or antiperspirant creams based on an oil-in-water emulsion which have, at 21° C., a viscosity of at least 50,000 mPa·s, preferably in the range from 200,000-1,500,000 mPa·s, i.e., they are in viscous to highly viscous paste form. These creams comprise wax components with a melting point of >50° C., nonionic water-in-oil emulsifiers, nonionic oil-in-water emulsifier with an HLB value of more than 7 and a polyol. Being soft creams, they can be applied either by using only the fingers, which is rejected by many consumers as being impractical, or by pouring the creams into special applicators, which are significantly more expensive than the stick sheaths for the deodorant or antiperspirant sticks according to the invention. If, after the heating and mixing, the compositions disclosed in DE 199 62 878 A1 and DE 199 62 881 A1 were left to cool statically, i.e., without stirring, then stick-like compositions would be obtained which have overall unfavorable application properties, such as poor hardness and/or inadequate stability, for example, as a result of phase separation or the formation of water condensation since the emulsifiers and the oils are not matched to one another as in the present invention.

### BRIEF SUMMARY OF THE INVENTION

Therefore, the object was to develop a deodorant or antiperspirant composition which is suitable as an effective carrier for water-soluble active ingredients and permits the rapid release of the active ingredient on the skin.

A further object was to develop a deodorant or antiperspirant composition with excellent cosmetic care properties.

A further object was to develop a deodorant or antiperspirant stick which, on the one hand, has high stability, i.e., strength, but on the other hand, has a pleasant release behavior, i.e., is not too solid but can be readily spread over the skin and in so doing releases an adequate amount of product.

A further object was to develop a deodorant or antiperspirant composition which, when applied to the skin, leaves behind as few as possible sticky or visible residues.

A further object was to develop a deodorant or antiperspirant composition which leaves behind as few as possible visible residues on clothing which comes into contact with the treated skin.

A further object was to develop a deodorant or antiperspirant composition which can be readily washed off from the skin.

A further object was to develop a deodorant or antiperspirant composition with a cost-performance ratio which is favorable economically and in terms of application.

Surprisingly and unforeseeably to the person skilled in the art, these objects were achieved through the subject-matter of the present application, namely a deodorant or antiperspirant stick in the form of an oil-in-water dispersion comprising
a) at least one lipid or wax component with a melting point of >50° C. which is not included in components b) or c),
b) at least one nonionic oil-in-water emulsifier with an HLB value of more than 7 within an oil-in-water emulsifier mixture with an average HLB value in the range from 10-19,
c) at least one nonionic water-in-oil emulsifier with an HLB value greater than 1.0 and less than or equal to 7.0, which can form liquid crystal structures with water on its own or with water in the presence of a hydrophilic emulsifier, as consistency regulator and/or water binder,
d) at least one oil liquid at 20° C., which does not constitute a fragrance component or an essential oil, where the (average) solubility parameter of the totality of the oils present d) in the presence of linear saturated fatty alcohols having at least 8 carbon atoms deviates by at most −0.7 (cal/cm³)^{0.5} or at most +0.7 (cal/cm³)^{0.5}, and in the presence of water-in-oil emulsifiers which are different from linear saturated fatty alcohols having at least 8 carbon atoms, in the absence of linear saturated fatty alcohols having at least 8 carbon atoms by at most −0.4 (cal/cm³)^{0.5} or at most +0.7 (cal/cm³)^{0.5} from the (average) solubility parameter of the water-in-oil emulsifier/the water-in-oil emulsifiers c),
e) at least one water-soluble polyhydric C₂-C₉-alkanol with 2-6 hydroxyl groups and/or at least one water-soluble polyethylene glycol with 3-20 ethylene oxide units,
f) 5 to less than 50% by weight of water, based on the overall composition,
g) at least one deodorant or antiperspirant active ingredient, where the stick has
h) a penetration force value in the range from 150-800 gram-force (g-force) at a penetration depth of 5,000 mm and
i) an electrical resistance of at most 300 kΩ (kiloohm).

### BRIEF DESCRIPTION OF THE SEVERAL VIEWS OF THE DRAWING(S)

Not Applicable

### DETAILED DESCRIPTION OF THE INVENTION

The lipid or wax component with a melting point of >50° C. forms a gel matrix with the oil(s) and optionally further higher-melting lipid or wax components; this gel matrix can absorb relatively large amounts of water and polyol. These structures, which are stabilized by certain amounts of water-in-oil emulsifiers and oil-in-water emulsifiers, leave behind a fresh, cooling impression upon application due to their water content. Here, the emulsifiers are matched to one another so that the stick compositions according to the invention are present in the form of an oil-in-water dispersion. Without wishing to be bound to this theory, it is assumed that the oil-in-water emulsifiers, together with some of the water-in-oil emulsifiers, form lamellar liquid crystal phases which are built up with some of the water to give a hydrophilic gel phase. This hydrophilic gel phase surrounds the aqueous bulk phase. Dispersed within this aqueous bulk phase are, in turn, the lipophilic components, surrounded by a lipophilic gel phase which is formed by the water-in-oil emulsifiers with some of the oil-in-water emulsifiers and some water.

The antiperspirant active ingredient is dissolved in the outer, continuous aqueous phase, resulting in a considerably improved and more efficient active ingredient release compared to the known anhydrous suspension sticks and water-in-oil emulsion sticks. This active ingredient release can be determined indirectly very readily by measuring the electrical resistance of the particular product. The precise measurement set-up and the measurement procedure are described below (see below). The sticks according to the invention accordingly have an electrical resistance of at most 300 kΩ, preferably of at most 100 kΩ and, particularly, preferably of at most 80 kΩ. By contrast, the sticks disclosed in WO 98/17238 A1 have an electrical resistance of more than 3,000 kΩ.

The solidification of the deodorant or antiperspirant sticks according to the invention does not take place on the basis of soap gels or fatty acid salt gels, fatty acids being understood as meaning alkanoic, alkenoic and alkinoic acids having at least 4 carbon atoms, which are substituted, for example, by hydroxyl groups. In a particularly preferred embodiment, the deodorant or antiperspirant sticks according to the invention are free of soap gels or fatty acid salt gels, in particular, free of lithium, sodium, potassium, ammonium, diethanolamine and triethanolamine salts of fatty acids.

The solidification of the deodorant or antiperspirant sticks according to the invention does not take place on the basis of inorganic and/or organic polymeric hydrogel formers, such as celluloses, cellulose derivatives, for example, hydroxyalkylcelluloses, polyacrylates, veegum or bentones. In a particularly preferred embodiment, the deodorant or antiperspirant sticks according to the invention are free of gels formed by inorganic and/or organic polymeric hydrogel formers.

Besides the favorable active ingredient release, the formulation as oil-in-water dispersion is accompanied by further advantages. First, the composition can be readily washed off from the skin. Second, during or following application to the skin, an attending oil-in-water cream forms together with the skin moisture.

Surprisingly and unexpectedly to the person skilled in the art, it has been found that the oil components and the water-in-oil emulsifier or the water-in-oil emulsifier mixture have to be matched to one another with regard to their solubility parameters in order to form stick compositions with satisfactory performance-related hardnesses. For a definition of the solubility parameter within the meaning of the present invention, reference is made to the publication “Solubility—Effects in Product, Package, Penetration and Preservation,” by Chr. D. Vaughan in Cosmetics & Toiletries, vol. 103, October 1988, pages 47-69. The values for the solubility parameters published therein are noted in the non-SI unit (cal/cm³)^{0.5}. For the sake of simplicity, the intention is to retain this non-SI unit in this specification.

In the stick compositions according to the invention, the (average) solubility parameter of the totality of the oils present in the presence of linear saturated fatty alcohols having a chain length of at least 8 carbon atoms deviates by at most −0.7 (cal/cm³)^{0.5} or at most +0.7 (cal/cm³)^{0.5}, preferably by at most −0.6 (cal/cm³)^{0.5} or at most +0.6 (cal/cm³)^{0.5}, particularly preferably by at most −0.4 (cal/cm³)^{0.5} or at most +0.5 (cal/cm³)^{0.5}, and in the presence of water-in-oil emulsifiers which differ from linear saturated fatty alcohols having a chain length of at least 8 carbon atoms, in the absence of linear saturated fatty alcohols with a chain length of at least 8 carbon atoms by at most −0.4 (cal/cm³)^{0.5} or at most +0.7 (cal/cm³)^{0.5}, preferably by at most −0.3 (cal/cm³)^{0.5} or at most +0.6 (cal/cm³)^{0.5}, particularly preferably by at most −0.2 (cal/cm³)^{0.5} or at most +0.5 (cal/cm³)^{0.5}, from the (average) solubility parameter of the water-in-oil emulsifier/water-in-oil emulsifiers. If water-in-oil emulsifier mixtures or oil mixtures are used, the average solubility parameter of the mixture is considered in each case, with the arithmetic mean according to the weight fraction of the individual components being considered. Within the scope of the invention, it is also possible for a weight fraction of the used oils liquid at 20° C. to consist of up to 20% by weight of oils whose solubility parameter deviates by more than −0.4 or −0.7 (cal/cm³)^{0.5} or by more than +0.7 (cal/cm³)^{0.5} from the (average) solubility parameter of the water-in-oil emulsifier (mixture). In a particularly preferred embodiment of the invention, no oils liquid at 20° C. are present whose solubility parameter deviates by more than ±1.0 (cal/cm³)^{0.5}, preferably by ±0.7 (cal/cm³)^{0.5} and particularly preferably by ±0.5 (cal/cm³)^{0.5} (average) solubility parameter of the water-in-oil emulsifier/water-in-oil emulsifiers.

The lipid or wax matrix of the stick compositions according to the invention comprises at least one lipid or wax component with a melting point of >50° C., which is not included in the nonionic oil-in-water emulsifiers with an HLB value of more than 7 or the nonionic water-in-oil emulsifiers with an HLB value greater than 1.0 and less than or equal to 7.0 which can form liquid crystal structures with water on its own or with water in the presence of a hydrophilic emulsifier.

Generally, waxes are of solid to brittle consistency, coarse to finely crystalline, transparent to opaque, but not glass-like, and melt above 50° C. without decomposition. Just a little above the melting point they are of low viscosity and exhibit a heavily temperature-dependent consistency and solubility.

According to the invention, preference is given, for example, to natural vegetable waxes, e.g., candelilla wax, carnauba wax, Japan wax, sugar cane wax, ouricoury wax, cork wax, sunflower wax, fruit waxes, such as orange waxes, lemon waxes, grapefruit wax, and animal waxes, e.g., beeswax, shellac wax and spermaceti. For the purposes of the invention, it may be particularly preferred to use hydrogenated or hardened waxes. Wax components which can be used are also chemically modified waxes, in particular, the hard waxes, such as, for example, montan ester waxes, hydrogenated jojoba waxes and sasol waxes. Synthetic waxes, which are likewise preferred according to the invention, include, for example, polyalkylene waxes and polyethylene glycol waxes, C₂₀-C₄₀-dialkyl esters of dimer acids, C₃₀₋₅₀-alkyl beeswax and alkyl and alkylaryl esters of dimer fatty acids.

A particularly preferred wax component is chosen from at least one ester of a saturated monohydric C₁₆-C₆₀-alcohol and a saturated C₈-C₃₆-monocarboxylic acid. According to the invention these also include lactides, the cyclic double esters of α-hydroxycarboxylic acids of the corresponding chain length. Esters of fatty acids and long-chain alcohols have proven particularly advantageous for the composition according to the invention because they impart excellent sensory properties to the antiperspirant preparation and high stability to the stick overall. The esters are composed of saturated, branched or unbranched monocarboxylic acids and saturated, branched or unbranched monohydric alcohols. According to the invention, it is also possible to use esters of aromatic carboxylic acids or hydroxycarboxylic acids (e.g., 12-hydroxystearic acid) and saturated, branched or unbranched alcohols if the wax component has a melting point of >50° C. It is particularly preferred to choose the wax components from the group of esters of saturated, branched or unbranched alkanecarboxylic acids of chain length from 12 to 24 carbon atoms and the saturated, branched or unbranched alcohols of chain length from 16 to 50 carbon atoms which have a melting point of >50° C.

In particular, C₁₆₋₃₆-alkyl stearates and C₁₈₋₃₆-alkyl hydroxystearoylstearates, C₂₀₋₄₀-alkyl erucates and cetearyl behenate may be advantageous as the wax component. The wax or the wax components have a melting point of >50° C., preferably >60° C.

A particularly preferred embodiment of the invention comprises a C₂₀-C₄₀-alkyl stearate as wax component. This ester is known under the name KesterwachsÒ K82H or Kesterwachs® K80H and is sold by Koster Keunen, Inc. It is the synthetic imitation of the monoester fraction of beeswax and is characterized by its hardness, its oil gelability and its broad compatibility with lipid components. This wax can be used as stabilizer and as consistency regulator for W/O and O/W emulsions. Kesterwachs offers the advantage that, even in low concentrations, it has excellent oil gelability and thus does not make the stick mass too heavy and allows a velvety release. A further particularly preferred embodiment of the invention comprises, as the wax component, cetearyl behenate, i.e., mixtures of cetyl behenate and stearyl behenate. This ester is known under the name KesterwachsÒ K62 and is sold by Koster Keunen, Inc.

Further preferred lipid or wax components with a melting point of >50° C. are the triglycerides of saturated and optionally hydroxylated C₁₂₋₃₀-fatty acids, such as hydrogenated triglyceride fats (hydrogenated palm oil, hydrogenated coconut oil, hydrogenated castor oil), glyceryl tribehenate (tribehenin) or glyceryl tri-12-hydroxystearate, also synthetic complete esters of fatty acids and glycols or polyols having 2-6 carbon atoms so long as they have a melting point above 50° C., for example, preferably C₁₈-C₃₆ acid triglyceride (Syncrowax® HGL-C).

According to the invention, hydrogenated castor oil, obtainable, e.g., as the commercial product Cutina® HR, is particularly preferred as the wax component.

Further preferred lipid or wax components with a melting point of >50° C. are the saturated linear C₁₄-C₃₆-carboxylic acids, in particular myristic acid, palmitic acid, stearic acid and behenic acid, and mixtures of these compounds, e.g., Syncrowax® AW 1C (C₁₈-C₃₆-fatty acids) or Cutina® FS 45 (palmitic and stearic acid).

Preferred deodorant or antiperspirant sticks according to the invention are characterized in that the lipid or wax component a) is chosen from esters of a saturated, monohydric C₁₆-C₆₀-alkanol and a saturated C₈-C₃₆-monocarboxylic acid, in particular, cetyl behenate, stearyl behenate and C₂₀-C₄₀-alkyl stearate, glycerol triesters of saturated linear C₁₂-C₃₀-carboxylic acids, which may be hydroxylated, candelilla wax, carnauba wax, beeswax, saturated linear C₁₄-C₃₆-carboxylic acids, and mixtures of the above-mentioned substances.

Further preferred deodorant or antiperspirant sticks according to the invention are characterized in that the lipid or wax component(s) a) is present overall in amounts of 4-20% by weight, preferably 8-15% by weight, based on the overall composition. In a particularly preferred embodiment, the ester/esters of a saturated, monohydric C₁₆-C₆₀-alcohol and a saturated C₈-C₃₆-monocarboxylic acid, which represents/represent the lipid or wax component(s) a), is/are present in amounts of 2-10% by weight, preferably 2-6% by weight, based on the overall composition.

The stick compositions according to the invention comprise at least one nonionic oil-in-water emulsifier with an HLB value of more than 7. These are emulsifiers generally known to the person skilled in the art, as listed, for example, in Kirk-Othmer, “Encyclopedia of Chemical Technology”, 3rd edition, 1979, volume 8, page 913-916. For ethoxylated products, the HLB value is calculated according to the formula HLB=(100-L):5, where L is the weight fraction of the lipophilic groups, i.e., of the fatty alkyl or fatty acyl groups, in the ethylene oxide adducts, expressed in percent by weight.

In selecting nonionic oil-in-water emulsifiers suitable according to the invention, it is particularly preferred to use a mixture of nonionic oil-in-water emulsifiers in order to be able to optimally adjust the stability of the stick compositions according to the invention. Here, the individual emulsifier components make a contribution to the overall HLB value or average HLB value of the oil-in-water emulsifier mixture according to their quantitative proportion of the total amount of the oil-in-water emulsifiers.

According to the invention, the average HLB value of the oil-in-water emulsifier mixture is 10-19, preferably 12-18 and particularly preferably 14-17. In order to achieve such average HLB values, oil-in-water emulsifiers from the HLB value ranges 10-14, 14-16 and optionally 16-19 are preferably combined with one another. The oil-in-water emulsifier mixtures can, of course, also comprise nonionic emulsifiers with HLB values in the range from >7-10 and 19-20; such emulsifier mixtures may likewise be preferred according to the invention. However, in another preferred embodiment, the deodorant or antiperspirant sticks according to the invention can also comprise just one oil-in-water emulsifier with an HLB value in the range from 10-19.

Preferred deodorant or antiperspirant sticks according to the invention are characterized in that the nonionic oil-in-water emulsifiers b) are chosen from ethoxylated C₈-C₂₄-alkanols with, on average, 10-100 mol of ethylene oxide per mole, ethoxylated C₈-C₂₄-carboxylic acids with, on average, 10-100 mol of ethylene oxide per mole, silicone copolyols with ethylene oxide units or with ethylene oxide and propylene oxide units, alkyl mono- and oligoglycosides having 8 to 22 carbon atoms in the alkyl radical, and ethoxylated analogs thereof, ethoxylated sterols, partial esters of polyglycerols with n=2 to 10 glycerol units and esterified with 1 to 4 saturated or unsaturated, linear or branched, optionally hydroxylated C₈-C₃₀-fatty acid radicals, if they have an HLB value of more than 7, and mixtures of the above-mentioned substances.

The ethoxylated C₈-C₂₄-alkanols have the formula R¹O(CH₂CH₂O)ₙH, where R¹ is a linear or branched alkyl and/or alkenyl radical having 8-24 carbon atoms and n, the average number of ethylene oxide units per molecule, is from 10-100, preferably 10-30 mol of ethylene oxide per 1 mol of caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical-grade mixtures thereof. Adducts of 10-100 mol of ethylene oxide onto technical-grade fatty alcohols having 12-18 carbon atoms, such as, for example, coconut, palm, palm kernel or tallow fatty alcohol, are also suitable.

The ethoxylated C₈-C₂₄-carboxylic acids have the formula R¹O(CH₂CH₂O)ₙH, where R¹O is a linear or branched saturated or unsaturated acyl radical having 8-24 carbon atoms and n, the average number of ethylene oxide units per molecule, is from 10-100, preferably 10-30 mol of ethylene oxide onto 1 mol of caprylic acid, 2-ethylhexanoic acid, capric acid, lauric acid, isotridecanoic acid, myristic acid, cetyl acid, palmitoleic acid, stearic acid, isostearic acid, oleic acid, elaidic acid, petroselic acid, arachidic acid, gadoleic acid, behenic acid, erucic acid and brassidic acid, and technical-grade mixtures thereof. Adducts of 10-100 mol of ethylene oxide onto technical-grade fatty acids having 12-18 carbon atoms, such as coconut, palm, palm kernel or tallow fatty acids, are also suitable. Particular preference is given to PEG-50 monostearate, PEG-100 monostearate, PEG-50 monooleate, PEG-100 monooleate, PEG-50 monolaurate and PEG-100 monolaurate.

Particular preference is given to using the C₁₂-C₁₈-alkanols or the C₁₂-C₁₈-carboxylic acids having in each case 10-30 units of ethylene oxide per molecule, and mixtures of these substances, in particular Ceteth-12, Ceteth-20, Ceteth-30, Steareth-12, Steareth-20, Steareth-30, Laureth-12 and Beheneth-20.

In addition, C₈-C₂₂-alkyl mono- and oligoglycosides are preferably used. C₈-C₂₂-alkyl mono- and oligoglycosides constitute known standard commercial surfactants and emulsifiers. They are prepared, in particular, by reacting glucose or oligosaccharides with primary alcohols having 8-22 carbon atoms. With regard to the glycoside radical, both monoglycosides in which a cyclic sugar radical is bonded glycosidically to the fatty alcohol, and also oligomeric glycosides with a degree of oligomerization to about 8, preferably 1-2, are suitable. The degree of oligomerization here is a statistical average value which is based on a homolog distribution customary for such technical products. Products which are obtainable under the trademark Plantacare® comprise a glucosidically bonded C₈-C₁₆-alkyl group on an oligoglucoside radical whose average degree of oligomerization is 1-2. Particularly preferred C₈-C₂₂-alkyl mono- and oligoglycosides are chosen from octyl glucoside, decyl glucoside, lauryl glucoside, palmityl glucoside, isostearyl glucoside, stearyl glucoside, arachidyl glucoside and behenyl glucoside, and mixtures thereof. The acylglucamides derived from glucamine are also suitable as nonionic oil-in-water emulsifiers.

Ethoxylated sterols, in particular, ethoxylated soya sterols, also represent oil-in-water emulsifiers suitable according to the invention. The degree of ethoxylation must be greater than 5, preferably at least 10, in order to have an HLB value greater than 7. Suitable commercial products are, e.g., PEG-10 Soy Sterol, PEG-16 Soy Sterol and PEG-25 Soy Sterol.

In addition, partial esters of polyglycerols having 2 to 10 glycerol units and esterified with 1 to 4 saturated or unsaturated, linear or branched, optionally hydroxylated C₈-C₃₀-fatty acid radicals are preferably used if they have an HLB value of more than 7. Particular preference is given to diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Particularly preferred deodorant or antiperspirant sticks according to the invention are characterized in that the nonionic oil-in-water emulsifier b) is present in a total amount of 0.5-10% by weight, particularly preferably 1-4% by weight and extraordinarily preferably 1.5-3% by weight, based on the overall composition.

The stick compositions according to the invention further comprise at least one nonionic water-in-oil emulsifier with an HLB value greater than 1.0 and less than or equal to 7.0 which can form liquid crystal structures with water alone or with water in the presence of a hydrophilic emulsifier, as consistency regulator and/or water binder. The water-in-oil emulsifier/emulsifiers contributes/contribute primarily to the construction of the lipophilic gel phase which surrounds the dispersed lipid/wax/oil phase, as well as, albeit to a lesser extent, to the construction of the hydrophilic gel phase which stabilizes the aqueous phase. Suitable nonionic water-in-oil emulsifiers are principally emulsifiers with an HLB value greater than 1.0 and less than or equal to 7.0. Some of these emulsifiers are listed, for example, in Kirk-Othmer, “Encyclopedia of Chemical Technology”, 3rd edition, 1979, volume 8, page 913. For ethoxylated adducts, the HLB value, as already mentioned, can also be calculated.

Preferred water-in-oil emulsifiers are:
linear saturated alkanols having 12-30 carbon atoms, in particular, having 16-22 carbon atoms, in particular, cetyl alcohol, stearyl alcohol, arachidyl alcohol, behenyl alcohol and lanolin alcohol or mixtures of these alcohols, as are obtainable in the industrial hydrogenation of vegetable and animal fatty acids,
esters and, in particular, partial esters of a polyol having 2-6 carbon atoms and linear saturated and unsaturated fatty acids having 12-30, in particular 14-22, carbon atoms, which may be hydroxylated. Such esters or partial esters are, for example, the monoesters and diesters of glycerol or ethylene glycol or the monoesters of propylene glycol with linear saturated and unsaturated C₁₂-C₃₀-carboxylic acids, which may be hydroxylated, in particular, those with palmitic acid and stearic acid, the sorbitan mono-, di- or triesters of linear saturated and unsaturated C₁₂-C₃₀-carboxylic acids, which may be hydroxylated, in particular, those of myristic acid, palmitic acid, stearic acid or of mixtures of these fatty acids, the pentaerythrityl mono-, di-, tri- and tetraesters and the methylglucose mono- and diesters of linear, saturated and unsaturated C₁₂-C₃₀-carboxylic acids, which may be hydroxylated;
sterols, i.e., steroids which carry a hydroxyl group on the C3 atom of the steroid backbone and are isolated both from animal tissue (zoosterols, e.g., cholesterol, lanosterol) and also from plants (phytosterols, e.g., ergosterol, stigmasterol, sitosterol) and from fungi and yeasts (mycosterols) and which may have low degrees of ethoxylation (1-5 EO);
alkanols and carboxylic acids having in each case 8-24 carbon atoms, in particular having 16-22 carbon atoms, in the alkyl group and 1-4 ethylene oxide units per molecule, which have an HLB value greater than 1.0 and less than or equal to 7.0,
glycerol monoethers of saturated and/or unsaturated, branched and/or unbranched alcohols of chain length from 8-30, in particular 12-18, carbon atoms,
partial esters of polyglycerols where n=2 to 10 glycerol units and esterified with 1 to 5 saturated or unsaturated, linear or branched, optionally hydroxylated C₈-C₃₀-fatty acid radicals if they have an HLB value of less than or equal to 7,
and mixtures of the above-mentioned substances.

According to the invention, it may be preferred to use just one water-in-oil emulsifier. In another preferred embodiment, the compositions according to the invention comprise mixtures, in particular technical-grade mixtures, of at least two water-in-oil emulsifiers. A technical-grade mixture is understood, for example, as meaning a commercial product such as Cutina® GMS, which constitutes a mixture of glyceryl monostearate and glyceryl distearate.

Water-in-oil emulsifiers which can be used particularly advantageously are stearyl alcohol, cetyl alcohol, glyceryl monostearate, in particular, in the form of the commercial products Cutina® GMS and Cutina® MD (ex Cognis), glyceryl distearate, glyceryl monocaprate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol(2) stearyl ether (Steareth-2), Steareth-5, Oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, Soy Sterol, PEG-1 Soy Sterol, PEG-5 Soy Sterol, PEG-2 monolaurate and PEG-2 monostearate.

Preferred deodorant or antiperspirant sticks according to the invention are characterized in that the nonionic water-in-oil emulsifier c) with an HLB value greater than 1.0 and less than or equal to 7.0, which can form liquid crystalline structures with water on its own or with water in the presence of a hydrophilic emulsifier, is chosen from linear saturated C₁₂-C₃₀-alkanols, ethylene glycol mono- and diesters of linear, saturated and unsaturated C₁₂-C₃₀-carboxylic acids, which may be hydroxylated, glycerol mono- and diesters of linear, saturated and unsaturated C₁₂-C₃₀-carboxylic acids, which may be hydroxylated, propylene glycol mono- and diesters of linear, saturated and unsaturated C₁₂-C₃₀-carboxylic acids, which may be hydroxylated, sorbitan mono-, di- and triesters of linear, saturated and unsaturated C₁₂-C₃₀-carboxylic acids, which may be hydroxylated, pentaerythrityl mono-, di-, tri- and tetraesters of linear, saturated and unsaturated C₁₂-C₃₀-carboxylic acids, which may be hydroxylated, methylglucose mono- and diesters of linear, saturated and unsaturated C₁₂-C₃₀-carboxylic acids, which may be hydroxylated, sterols, alkanols and carboxylic acids having in each case 8-24 carbon atoms, in particular, having 16-22 carbon atoms, in the alkyl group and 1-4 ethylene oxide units per molecule, which have an HLB value of greater than 1.0 and less than or equal to 7.0, glycerol monoethers of saturated and/or unsaturated, branched and/or unbranched alcohols of chain length from 8-30, in particular 12-18, carbon atoms, partial esters of polyglycerols where n=2 to 10 glycerol units and esterified with 1 to 5 saturated or unsaturated, linear or branched, optionally hydroxylated C₈-C₃₀-fatty acid radicals if they have an HLB value of less than or equal to 7, and mixtures of the above-mentioned substances.

Particularly preferred deodorant or antiperspirant sticks according to the invention are characterized in that the nonionic water-in-oil emulsifier(s) c) with an HLB value greater than 1.0 and less than or equal to 7.0, which can form liquid crystalline structures with water on its own or with water in the presence of a hydrophilic emulsifier, is/are present in a total amount of 0.1-15% by weight, particularly preferably 0.5-8% by weight and extraordinarily preferably 1-4% by weight, based on the total weight of the composition. In addition, amounts of from 2-3% by weight, based on the total weight of the composition, may also be extraordinarily preferred according to the invention.

The stick compositions according to the invention further comprise at least one oil liquid at 20° C. which does not represent a fragrance component or an essential oil, where the (average) solubility parameter of the totality of the oils present in the presence of linear saturated fatty alcohols with a chain length of at least 8 carbon atoms deviates by at most −0.7 (cal/cm³)^{0.5} or at most +0.7 (cal/cm³)^{0.5}, preferably by at most −0.6 (cal/cm³)^{0.5} or at most +0.6 (cal/cm³)^{0.5}, particularly preferably, by at most −0.4 (cal/cm³)^{0.5} or at most +0.5 (cal/cm³)^{0.5}, and in the presence of water-in-oil emulsifiers which are different from linear saturated fatty alcohols with a chain length of at least 8 carbon atoms, in the absence of linear saturated fatty alcohols with a chain length of at least 8 carbon atoms by at most −0.4 (cal/cm³)^{0.5} or at most +0.7 (cal/cm³)^{0.5}, preferably by at most −0.3 (cal/cm³)^{0.5} or at most +0.6 (cal/cm³)^{0.5}, particularly preferably by at most −0.2 (cal/cm³)^{0.5} or at most +0.5 (cal/cm³)^{0.5}, from the (average) solubility parameter of the water-in-oil emulsifier/water-in-oil emulsifiers. The matching of the oil/oils used to the water-in-oil emulsifier(s) used constitutes an important parameter of this invention. Not matching the water-in-oil emulsifier and the oil component(s) from the point of view of the solubility parameter within the claimed limits gives sticks of inadequate hardness and stability for application.

Oils preferred according to the invention are chosen from branched saturated or unsaturated fatty alcohols having 6-30 carbon atoms. These alcohols are also often referred to as Guerbet alcohols since they are obtainable according to the Guerbet reaction. Preferred alcohol oils are hexyldecanol (Eutanol® G 16, Guerbitol® T 16), octyldodecanol (Eutanol® G, Guerbitol® 20), 2-ethylhexyl alcohol and the commercial products Guerbitol® 18, Isofol® 12, Isofol®16, Isofol 24, Isofol® 36, Isocarb® 12, Isocarb® 16 or Isocarb® 24.

Further preferred oil components are mixtures of Guerbet alcohols and Guerbet alcohol esters, e.g., the commercial product Cetiol® PGL (hexyldecanol and hexyldecyl laurate).

Further oils preferred according to the invention are chosen from the triglycerides of linear or branched, saturated or unsaturated, optionally hydroxylated C₈₋₃₀-fatty acids. The use of natural oils, e.g., soya oil, cotton seed oil, sunflower oil, palm oil, palm kernel oil, linseed oil, almond oil, castor oil, corn oil, olive oil, rapeseed oil, sesame oil, thistle oil, wheat germ oil, peach kernel oil and the liquid fractions of coconut oil and the like may be particularly suitable. Also suitable, however, are synthetic triglyceride oils, in particular capric/caprylic triglycerides, e.g., the commercial products Myritol® 318, Myritol® 331 (Cognis) or Miglyol® 812 (Mils) with unbranched fatty acid radicals and glyceryl triisostearin and the commercial products Estol® GTEH 3609 (Uniqema) or Myritol® GTEH (Cognis) with branched fatty acid radicals.

Further oils particularly preferred according to the invention are chosen from the dicarboxylic acid esters of linear or branched C₂-C₁₀alkanols, in particular, diisopropyl adipate, di-n-butyl adipate, di(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di(2-hexyldecyl)succinate.

Further oils particularly preferred according to the invention are chosen from the addition products of from 1 to 5 propylene oxide units onto mono- or polyhydric C₈₋₂₂-alkanols, such as octanol, decanol, decanediol, lauryl alcohol, myristyl alcohol and stearyl alcohol, e.g., PPG-2 myristyl ether and PPG-3 myristyl ether (Witconol® APM).

For the use of the oils listed below in the stick compositions according to the invention it should be ensured that their fraction of the overall oil mixture is only of a size such that the average solubility parameter of the overall oil mixture, as claimed according to the invention and described above, is matched to the average solubility parameter of the water-in-oil emulsifier. Corresponding oils are chosen from the esters of the linear or branched saturated or unsaturated fatty alcohols having 2-30 carbon atoms with linear or branched saturated or unsaturated fatty acids having 2-30 carbon atoms, which may be hydroxylated. These include hexyldecyl stearate (Eutanol® G 16 S), hexyldecyl laurate, isodecyl neopentanoate, isononyl isononanoate, 2-ethylhexyl palmitate (Cegesoft® C 24) and 2-ethylhexyl stearate (Cetiol® 868). Likewise of limited suitability are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and dipalmitate.

Further oils which can be used with consideration to the solubility parameter harmonization only in small amounts, if at all, are chosen from the addition products of at least 6 ethylene oxide and/or propylene oxide units onto mono- or polyhydric C₃₋₂₂-alkanols, such as butanol, butanediol, myristyl alcohol and stearyl alcohol, e.g., PPG-14 butyl ether (Ucon Fluid® AP), PPG-9 butyl ether (Breox® B25), PPG-10 butanediol (Macol® 57) and PPG-15 stearyl ether (Arlamol® E).

Further oils which can be used with consideration to the solubility parameter harmonization only in small amounts, if at all, are chosen from the C₈-C₂₂-fatty alcohol esters of monobasic or polybasic C₂-C₇-hydroxycarboxylic acids, in particular, the esters of glycolic acid, lactic acid, malic acid, tartaric acid, citric acid and salicylic acid. Such esters based on linear C_{14/15}-alkanols, e.g., C₁₂-C₁₈-alkyl lactate, and on C_{12/13}-alkanols branched in the 2 position are available under the trade name Cosmacol® from Nordmann, Rassmann GmbH & Co., Hamburg, in particular the commercial products Cosmacol® ESI, Cosmacol® EMI and Cosmacol® ETI.

Further oils which can be used with consideration to the solubility parameter harmonization only in small amounts, if at all, are chosen from the symmetrical, asymmetrical or cyclic esters of carbonic acid with fatty alcohols, e.g., glycerol carbonate, dicaprylyl carbonate (Cetiol® CC) or the esters of DE 197 56 454 A1.

Further oils which can be used with consideration to the solubility parameter harmonization only in small amounts, if at all, are chosen from the esters of dimers of unsaturated C₁₂-C₂₂-fatty acids (dimer fatty acids) with monohydric linear, branched or cyclic C₂-C₁₈-alkanols or with polyhydric linear or branched C₂-C₆-alkanols.

According to the invention; it may be preferred to use mixtures of the above-mentioned oils.

Preferred deodorant or antiperspirant sticks according to the invention are characterized in that the oil d) liquid at 20° C. is chosen from branched saturated or unsaturated fatty alcohols having 6-30 carbon atoms, triglycerides of linear or branched, saturated or unsaturated, optionally hydroxylated C₈₋₃₀-fatty acids, dicarboxylic acid esters of linear or branched C₂-C₁₀-alkanols, esters of branched saturated or unsaturated fatty alcohols having 2-30 carbon atoms with linear or branched saturated or unsaturated fatty acids having 2-30 carbon atoms, which may be hydroxylated, addition products of from 1 to 5 propylene oxide units onto mono- or polyhydric C₈₋₂₂-alkanols, addition products of at least 6 ethylene oxide and/or propylene oxide units onto mono- or polyhydric C₃₋₂₂-alkanols, C₈-C₂₂-fatty alcohol esters of monobasic or polybasic C₂-C₇-hydroxycarboxylic acids, symmetrical, asymmetrical or cyclic esters of carbonic acid with fatty alcohols, the esters of dimers of unsaturated C₁₂-C₂₂-fatty acids (dimer fatty acids) with monohydric linear, branched or cyclic C₂-C₁₈-alkanols or with polyhydric linear or branched C₂-C₆-alkanols, and mixtures of the above-mentioned substances.

Particularly preferred deodorant or antiperspirant sticks according to the invention are characterized in that the oil(s) d) liquid at 20° C. is/are present in a total amount of 3-20% by weight, preferably 5-14% by weight, based on the total weight of the composition.

In a further particularly preferred embodiment of the invention, the fraction of oil(s) whose solubility parameter deviates by more than −0.4 or −0.7 (cal/cm³)^{0.5} or by more than +0.7 (cal/cm³)^{0.5} from the average value of the water-in-oil emulsifiers used, is at most 20% by weight, based on the total weight of oils liquid at 20° C. In a further particularly preferred embodiment of the invention, no oils liquid at 20° C. are present whose solubility parameter deviates by more than ±1.0 (cal/cm³)^{0.5} from the (average) solubility parameter of the water-in-oil emulsifier/the water-in-oil emulsifiers c).

Corresponding less suitable or (depending on the water-in-oil emulsifier used) even unsuitable oil components are, for example, silicone oils and hydrocarbon oils.

Silicone oils, which include, for example, dialkyl- and alkylarylsiloxanes, such as, for example, cyclopentasiloxane, cyclohexasiloxane, dimethylpolysiloxane and methylphenylpolysiloxane, but also hexamethyldisiloxane, octamethyltrisiloxane and decamethyltetrasiloxane, have solubility paramaters in the range from about 5.7 to 6.3 (cal/cm³)^{0.5}, which deviates by more than 0.4 (cal/cm³)^{0.5} from the value of most of the water-in-oil emulsifiers used according to the invention.

Natural and synthetic hydrocarbons, such as, for example, paraffin oils, isohexadecane, isoeicosane, polyisobutenes or polydecenes, which are available, for example, under the name Emery® 3004, 3006, 3010 or under the name Ethylfio® from Albemarle or Nexbase® 2004G from Nestlé, and 1,3-di(2-ethylhexyl)cyclohexane (Cetiol® S) are likewise examples of oil components which are less preferred according to the invention.

In a preferred embodiment of the invention, the proportion of the silicone oils and/or hydrocarbons should not be greater than 20%, based on the total weight of oils liquid at 20° C., otherwise the sticks according to the invention do not achieve the desired hardness and stability for application. In a particularly preferred embodiment of the invention, no silicone oils and/or hydrocarbons are present.

The stick compositions according to the invention further comprise at least one water-soluble polyhydric C₂-C₉-alkanol with 2-6 hydroxyl groups and/or at least one water-soluble polyethylene glycol with 3-20 ethylene oxide units, and mixtures thereof. These components are preferably chosen from 1,2-propylene glycol, 2-methyl-1,3-propanediol, glycerol, butylene glycols, such as 1,2-butylene glycol, 1,3-butylene glycol and 1,4-butylene glycol, pentylene glycols, such as 1,2-pentanediol and 1,5-pentanediol, hexanediol, such as 1,6-hexanediol, hexanetriols, such as 1,2,6-hexanetriol, 1,2-octanediol, 1,8-octanediol, dipropylene glycol, tripropylene glycol, diglycerol, triglycerol, erythritol, sorbitol, and mixtures of the above-mentioned substances. Suitable water-soluble polyethylene glycols are chosen from PEG-3, PEG-4, PEG-6, PEG-7, PEG-8, PEG-9, PEG-10, PEG-12, PEG-14, PEG-16, PEG-18 and PEG-20, and mixtures thereof, preference being given to PEG-3 to PEG-8. Sugars and certain sugar derivatives, such as fructose, glucose, maltose, maltitol, mannitol, inositol, sucrose, trehalose and xylose are also suitable according to the invention.

Preferred deodorant or antiperspirant sticks according to the invention are characterized in that the at least one water-soluble polyhydric C₂-C₉-alkanol with 2-6 hydroxyl groups and/or at least one water-soluble polyethylene glycol with 3-20 ethylene oxide units is chosen from 1,2-propylene glycol, 2-methyl-1,3-propanediol, glycerol, butylene glycols, such as 1,2-butylene glycol, 1,3-butylene glycol and 1,4-butylene glycol, pentylene glycols, such as 1,2-pentanediol and 1,5-pentanediol, hexanediols, such as 1,6-hexanediol, hexanetriols, such as 1,2,6-hexanetriol, 1,2-octanediol, 1,8-octanediol, dipropylene glycol, tripropylene glycol, diglycerol, triglycerol, erythritol, sorbitol, and mixtures of the above-mentioned substances.

Particularly preferred deodorant or antiperspirant sticks according to the invention are characterized in that the at least one water-soluble polyhydric C₂-C₉-alkanol with 2-6 hydroxyl groups and/or at least one water-soluble polyethylene glycol with 3-20 ethylene oxide units is present overall in amounts of 3-25% by weight, preferably 8-18% by weight, based on the overall composition.

The proportion of water in the composition according to the invention is 5 to less than 50% by weight, preferably 10 to less than 30% by weight, particularly preferably 15-28% by weight, extraordinarily preferably 20-26% by weight, in each case based on the overall composition.

The stick compositions according to the invention further comprise at least one deodorant and/or antiperspirant active ingredient.

Deodorant active ingredients preferred according to the invention are odor absorbers, deodorizing ion exchangers, antimicrobial agents, prebiotically effective components, and enzyme inhibitors or, particularly preferably, combinations of said active ingredients.

Silicates serve as odor absorbers which also simultaneously and advantageously aid the rheological properties of the composition according to the invention. Silicates which are particularly advantageous according to the invention include, in particular, phyllosilicates and, among these, in particular montmorillonite, kaolinite, ilite, beidellite, nontronite, saponite, hectorite, bentonite, smectite and talc. Further advantageous odor absorbers are, for example, zeolites, zinc ricinoleate, cyclodextrins, certain metal oxides, such as, for example, aluminum oxide, and chlorophyll. They are preferably used in an amount of from 0.1-10% by weight, particularly preferably 0.5-7% by weight and extraordinarily preferably 1-5% by weight, in each case based on the overall composition.

Antimicrobial active ingredients are understood according to the invention as meaning active ingredients which reduce the number of skin microbes involved in odor formation, or inhibit their growth. These microbes include, inter alia, various species from the group of staphylococci, the group of corynebacteria, anaerococci and micrococci.

According to the invention, preferred antimicrobial active ingredients are, in particular, organohalogen compounds and halides, quaternary ammonium compounds, a number of plant extracts and zinc compounds. These include, inter alia, triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphen bromide, ammonium phenolsulfonate, benzalkonium halides, benzalkonium cetyl phosphate, benzalkonium saccharinates, benzethonium chloride, cetylpyridinium chloride, laurylpyridinium chloride, laurylisoquinolinium bromide, methylbenzedonium chloride. It is also possible to use phenol, phenoxyethanol, disodium dihydroxyethylsulfosuccinylundecylenate, sodium bicarbonate, zinc lactate, sodium phenolsulfonate and zinc phenolsulfonate, ketoglutaric acid, terpene alcohols, such as, for example, farnesol, chlorophyllin-copper complexes, α-monoalkyl glycerol ether with a branched or linear saturated or unsaturated, optionally hydroxylated C₆-C₂₂-alkyl radical, particularly preferably α-(2-ethylhexyl)glycerol ether, commercially available as Sensiva® SC 50 (ex Schülke & Mayr), carboxylic esters of mono-, di- and triglycerol (e.g., glycerol monolaurate, diglycerol monocaprate), lantibiotics, and plant extracts (e.g., green tea and constituents of linden blossom oil).

Further preferred deodorant active ingredients are chosen from so-called prebiotically effective components which, according to the invention, are understood as meaning those components which inhibit only or at least predominantly the odor-forming microbes of the skin microflora, but not the desired, i.e., the non-odor-forming microbes, which belong to a healthy skin microflora. The active ingredients which are disclosed in the laid-open specifications DE 10333245 and DE 10 2004 011 968 as being prebiotically effective are explicitly included here; these include conifer extracts, in particular from the group of Pinaceae, and plant extracts from the group of Sapindaceae, Araliaceae, Lamiaceae and Saxifragaceae, in particular, extracts from Picea spp., Paullinia sp., Panax sp., Lamium album or Ribes nigrum, and mixtures of these substances.

Further preferred deodorant active ingredients are chosen from the antimicrobially effective perfume oils and the deosafe perfume oils which are obtainable from Symrise, formerly Haarmann and Reimer.

The enzyme inhibitors include substances which inhibit the enzymes responsible for the decomposition of sweat, in particular, arylsulfatase, β-glucuronidase, aminoacylase, esterases, lipases and/or lipoxygenase, e.g., trialkylcitric esters, in particular, triethyl citrate, or zinc glycinate.

Preferred deodorant or antiperspirant sticks according to the invention are characterized in that the at least one deodorant active ingredient is chosen from arylsulfatase inhibitors, β-glucuronidase inhibitors, aminoacylase inhibitors, esterase inhibitors, lipase inhibitors and lipoxygenase inhibitors, α-monoalkyl glycerol ethers having a branched or linear saturated or unsaturated, optionally hydroxylated C₆-C₂₂-alkyl radical, in particular α-(2-ethylhexyl) glycerol ether, phenoxyethanol, antimicrobial perfume oils, deosafe perfume oils, prebiotically effective components, trialkylcitric esters, in particular, triethyl citrate, active ingredients which reduce the number of skin microbes involved in odor formation from the group of staphylococci, corynebacteria, anaerococci and micrococci or inhibit their growth, zinc compounds, in particular zinc phenolsulfonate and zinc ricinoleate, organohalogen compounds, in particular triclosan, chlorhexidine, chlorhexidine gluconate and benzalkonium halides, quatemary ammonium compounds, in particular cetylpyridinium chloride, odor absorbers, in particular silicates and zeolites, sodium bicarbonate, lantibiotics, and mixtures of the above-mentioned substances.

Preferred deodorant or antiperspirant sticks according to the invention are characterized in that the at least one deodorant active ingredient is present in a total amount of 0.1-10% by weight, preferably 0.2-7% by weight, particularly preferably, 0.3-5% by weight and extraordinarily preferably 0.4-1.0% by weight, based on the total weight of the active substance in the overall composition.

Preferred deodorant or antiperspirant sticks according to the invention are characterized in that at least one antiperspirant active ingredient chosen from the water-soluble astringent inorganic and organic salts of aluminum, zirconium and zinc and any mixtures of these salts is present. Particularly preferred antiperspirant active ingredients are chosen from the aluminum chlorhydrates, in particular, the aluminum chlorhydrates with the general formula [Al₂(OH)₅Cl.2-3H₂O]ₙ, which may be present in nonactivated or inactivated (depolymerized) form, also aluminum sesquichlorhydrate, aluminum chlorohydrex-propylene glycol (PG) or -polyethylene glycol (PEG), aluminum sesquichlorohydrex-PG or -PEG, aluminum-PG-dichlorohydrex or aluminum-PEG-dichiorohydrex, aluminum hydroxide, also chosen from the aluminum zirconium chlorhydrates, such as aluminum zirconium trichlorhydrate, aluminum zirconium tetrachlorhydrate, aluminum zirconium pentachlorhydrate, aluminum zirconium octachlorhydrate, the aluminum-zirconium chlorhydrate glycine complexes, such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂.12H₂O, alum), aluminum undecylenoyl collagen amino acid, sodium aluminum lactate +aluminum sulfate, sodium aluminum chlorohydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxyallantoinate, sodium-aluminum chlorohydroxylactate, zinc chloride, zinc sulfocarbolate, zinc sulfate and zirconium chlorhydrate. According to the invention, solubility in water is understood as meaning a solubility of at least 5% by weight at 20° C., i.e., at amounts of at least 5 g of the antiperspirant active ingredient are soluble in 95 g of water at 20° C. The antiperspirant active ingredients can be used as aqueous solutions.

Particularly preferred deodorant or antiperspirant sticks according to the invention are characterized in that the at least one antiperspirant active ingredient is present in an amount of 3-25% by weight, preferably 5-22% by weight and in particular 10-20% by weight, based on the total weight of the active substance in the overall composition. In a particularly preferred embodiment, the composition comprises an astringent aluminum salt, in particular, aluminum chlorhydrate, which is sold, for example, in powder form as Micro Dry® Ultrafine from Reheis, in the form of an aqueous solution as Locron® L from Clariant, as Chlorhydrol®, and in activated form as Reach® 501 from Reheis. An aluminum sesquichlorhydrate from Reheis supplied under the name Reach® 301 is likewise particularly preferred. The use of aluminum-zirconium-tetrachiorohydrex glycine complexes, which are available, for example, from Reheis under the name Rezal® 36G can also be particularly preferred according to the invention.

In a further particularly preferred embodiment, the stick compositions according to the invention can comprise both at least one deodorant active ingredient and also at least one antiperspirant active ingredient.

Particularly preferred deodorant or antiperspirant sticks according to the invention are characterized in that at least one lipid or wax component with a melting point in the range from 25-<50° C., chosen from coconut fatty acid glycerol mono-, di- and triesters, Butyrospermum Parkii (shea butter) and esters of saturated, monohydric C₈-C₁₈-alcohols with saturated C₁₂-C₁₈-monocarboxylic acids, and mixtures of these substances, is present. These lower-melting lipid or wax components permit a consistency optimization of the product and a minimization of the visible residues on the skin. Particular preference is given to commercial products with the INCI name Cocoglycerides, in particular, the commercial products Novata® (ex Cognis), particularly preferably Novata® AB, a mixture of C₁₂-C₁₈-mono-, di- and triglycerides, which melts in the range from 30-32° C., and the products of the Softisan series (Sasol Germany GmbH) with the INCI name Hydrogenated Cocoglycerides, in particular Softisan 100, 133, 134, 138, 142. Further preferred esters of saturated, monohydric C₁₂-C₁₈-alcohols with saturated C₁₂-C₁₈-monocarboxylic acids are stearyl laurate, cetearyl stearate (e.g., Crodamol® CSS), cetyl palmitate (e.g., Cutina® CP) and myristyl myristate (e.g. Cetiol® MM).

Further particularly preferred deodorant or antiperspirant sticks according to the invention are characterized in that the at least one lipid or wax component with a melting point in the range from 25-<50° C. is present in amounts of from 0.01 to 20% by weight, preferably 3-20% by weight, particularly preferably 5-18% by weight and extraordinarily preferably 6-15% by weight, based on the overall composition.

Particularly preferred deodorant or antiperspirant sticks according to the invention are characterized in that they further comprise at least one solid, water-insoluble particulate filler for improving the stick consistency and the sensory properties. In an extraordinarily preferred embodiment, this filler is chosen from optionally modified starches (e.g., from corn, rice, potatoes) and starch derivatives, which are pregelatinized if desired, in particular, starch derivatives of the DRY FLO® type, cellulose and cellulose derivatives, silicon dioxide, silicas, e.g., Aerosil® grades, spherical polyalkylsesquisiloxane particles (in particular, Aerosil® R972 and Aerosil® 200V from Degussa), silica gels, talc, kaolin, clays, e.g., bentonite, magnesium aluminum silicates, boron nitride, lactoglobulin derivatives, e.g., sodium C₈₋₁₆-isoalkylsuccinyllactoglobulin sulfonate, obtainable from Brooks Industries as commercial product Biopol® OE, glass powders, polymer powders, in particular, from polyolefins, polycarbonates, polyurethanes, polyamides, e.g., nylon, polyesters, polystyrenes, polyacrylates, (meth)acrylate- or (meth)acrylate-vinylidene copolymers, which may be crosslinked, or silicones, and mixtures of these substances.

Polymer powders based on a polymethacrylate copolymer are obtainable, for example, as commercial product Polytrap® 6603 (Dow Corning). Other polymer powders, e.g., based on polyamides, are available under the name Orgasol® 1002 (polyamide-6) and Orgasol® 2002 (polyamide-12) from Elf Atochem. Further polymer powders which are suitable for the purpose according to the invention are, for example, polymethacrylates (Micropearl® M from SEPPIC or Plastic Powder A from NIKKOL), styrene-divinylbenzene copolymers (Plastic Powder FP from NIKKOL), polyethylene and polypropylene powders (ACCUREL® EP 400 from AKZO) or else silicone polymers (Silicone Powder XZ-1605 from Dow Corning).

Particularly preferred deodorant or antiperspirant sticks according to the invention are characterized in that they comprise at least one solid, water-insoluble particulate filler in a total amount of from 0.01 to 20% by weight, preferably 5-15% by weight, in each case based on the overall composition.

Particularly preferred deodorant or antiperspirant sticks according to the invention are characterized in that in addition at least one fragrance component is present.

Fragrance components which can be used are perfumes, perfume oils or perfume oil constituents. According to the invention, perfume oils or fragrances can be individual fragrance compounds, e.g., the synthetic products of the ester, ether, aldehyde, ketone, alcohol and hydrocarbon type. Fragrance compounds of the ester type are, for example, benzyl acetate, phenoxyethyl isobutyrate, p-tert-butyl cyclohexylacetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramat, melusat and jasmecyclate. The ethers include, for example, benzyl ethyl ether and ambroxan. The aldehydes include, for example, the linear alkanals having 8-18 carbon atoms, citral, citronellal, citronellyloxyacetaldehyde, cyclamen aldehyde, lilial and bourgeonal. The ketones include, for example, the ionones, alpha-isomethylionone and methyl cedryl ketone. The alcohols include anethol, citronellol, eugenol, geraniol, linalool, phenyl ethyl alcohol and terpineol. The hydrocarbons include primarily the terpenes, such as limonene and pinene. However, preference is given to using mixtures of different fragrances which together produce a pleasant scent note.

Such perfume oils can also comprise natural fragrance mixtures, as are accessible from vegetable sources, e.g., pine oil, citrus oil, jasmine oil, patchouli oil, rose oil or ylang-ylang oil. Likewise suitable are clary sage oil, camomile oil, oil of cloves, melissa oil, mint oil, cinnamon leaf oil, linden blossom oil, juniper berry oil, vetiver oil, olibanum oil, galbanum oil and labdanum oil, and orange blossom oil, neroli oil, orange peel oil and sandalwood oil.

In order to be perceptible, a fragrance must be volatile where, besides the nature of the functional groups and the structure of the chemical compound, the molar mass also plays an important role. Thus, most fragrances have molar masses up to about 200 daltons, whereas molar masses of 300 daltons and above more likely represent an exception. On account of the varying volatility of fragrances, the odor of a perfume or scent composed of two or more fragrances changes during vaporization, the odor impressions being divided into “top note,” “middle note or body” and “end note or dryout.” Since the odor perception is based to a large degree also on the odor intensity, the top note of a perfume or fragrance consists not merely of readily volatile compounds, while the end note consists for the greatest part of less volatile, i.e., Tenacious, fragrances. When composing perfumes, more readily volatile fragrances can, for example, be bound to certain fixatives, as a result of which their excessively rapid vaporization is prevented. The division below of the fragrances into “more readily volatile” and “Tenacious” fragrances thus does not convey any information about the odor impression and whether the corresponding fragrance is perceived as a top note or middle note.

Tenacious fragrances which can be used for the purposes of the present invention are, for example, the essential oils, such as angelica root oil, anise oil, amica flower oil, basil oil, bay oil, bergamot oil, champaca flower oil, noble fir oil, noble fir cone oil, elemy oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calmus oil, camomile oil, camphor oil, canaga oil, cardamom oil, cassia oil, pine needle oil, copaiva balsam oil, coriander oil, spearmint oil, caraway oil, cumin oil, lavender oil, lemongrass oil, lime oil, mandarin oil, melissa oil, muscat kernel oil, myrrh oil, oil of cloves, neroli oil, niaouli oil, olibanum oil, orange oil, origanum oil, palmarosa oil, patchouli oil, peru balsam oil, petit grain oil, pepper oil, peppermint oil, pimento oil, pine oil, rose oil, rosemary oil, sandalwood oil, celery oil, spike oil, star anise oil, terpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil.

However, the higher-boiling or solid fragrances of natural or synthetic origin can also be used for the purposes of the present invention as Tenacious fragrances or fragrance mixtures, i.e., scents. These compounds include the compounds named below and mixtures thereof: ambrettolide, a-amylcinnamaldehyde, anethol, anise aldehyde, anise alcohol, anisole, methyl anthranilate, acetophenone, benzylacetone, benzaldehyde, ethyl benzoate, benzophenone, benzyl alcohol, benzyl acetate, benzyl benzoate, benzyl formate, benzyl valerate, bomeol, bornyl acetate, α-bromostyrene, n-decyl aldehyde, n-dodecyl aldehyde, eugenol, eugenol methyl ether, eucalyptol, famesol, fenchone, fenchyl acetate, geranyl acetate, geranyl formate, heliotropin, methyl heptyne carboxylate, heptaldehyde, hydroquinone dimethyl ether, hydroxycinnamaldehyde, hydroxycinnamal alcohol, indole, irone, isoeugenol, isoeugnol methyl ether, isosafrol, jasmone, camphor, carvacrol, carvone, p-cresol methyl ether, coumarin, p-methoxyacetophenone, methyl n-amyl ketone, methyl methylanthranilate, p-methylacetophenone, methylchavicol, p-methylquinoline, methyl β-naphthyl ketone, methyl-n-nonylacetaldehyde, methyl n-nonyl ketone, muscone, β-naphthol ethyl ether, β-naphthol methyl ether, nerol, nitrobenzene, n-nonylaldehyde, nonyl alcohol, n-octylaldehyde, p-oxyacetophenone, pentadecanolide, β-phenylethyl alcohol, phenylacetaldehyde dimethyl acetal, phenylacetic acid, pulegone, safrol, isoamyl salicylate, methyl salicylate, hexyl salicylate, cyclohexyl salicylate, santalol, skatole, terpineol, thymene, thymol, y-undelactone, vanillin, veratrum aldehyde, cinnamaldehyde, cinnamal alcohol, cinnamic acid, ethyl cinnamate and benzyl cinnamate.

The more readily volatile fragrances include, in particular, the lower-boiling fragrances of natural or synthetic origin which can be used alone or in mixtures. Examples of more readily volatile fragrances are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral, citronellal.

Particularly preferred deodorant or antiperspirant sticks according to the invention are characterized in that at least one fragrance component is present in a total amount of from 0.00001 to 4% by weight, preferably 0.5-2% by weight, in each case based on the overall composition.

The stick compositions according to the invention are also characterized by a penetration force value in the range from 150-800 gram-force (g-force), preferably 200-750 gram-force (g-force), particularly preferably 350-600 gram-force (g-force), at a penetration depth of 5,000 mm. The penetration force value is a measure of the hardness of a stick (and also of a solid cream composition) and indicates at what maximum force a defined measuring probe, here a stainless steel 45° cone (model TA 15) is inserted perpendicularly (axially) into the antiperspirant mass to be measured at a feed rate of 2 mm/second to a penetration depth suitable for the measurement, here to a penetration depth of 5,000 mm. The determination of the penetration force value is carried out using the TA-XT2i texture analyzer from Stable Micro Systems (Vienna Court, Lammas Road, Godalming, Surrey GU7 1YL). The maximum force is given in gram-force (9-force). Here, lower values indicate a softer composition, harder compositions have a higher penetration force value. Cream-like compositions are often measured with a penetration depth of 10,000 mm in order to obtain more precise values. In most cases this insertion depth cannot be measured for harder stick masses since the stick mass often starts to break. A doubling of the penetration depth means that the measurement value of the maximum force increases approximately threefold to fourfold. The measurements are carried out under ambient conditions of 30° C. and 50% relative atmospheric humidity, the sample temperature is 23° C. The antiperspirant creams disclosed in DE 199 62 878 A1 and DE 199 62 881 A1 have penetration force values of 9-15 gram-force (g-force) under the measurement conditions specified here.

The stick compositions according to the invention are also characterized by an electrical resistance of at most 300 kΩ. Preference is given to an electrical resistance of at most 100 kΩ, particularly preferably, of at most 80 kΩ. The resistance is measured using a multimeter voltcraft model VC820 with automatic measurement range switching (0-400 Ω/40MΩ (±1%+2 dgt)) and two 1.0 mm stainless steel microtip probes. The distance between the electrodes is fixed using a millimeter gauge. The measurement is carried out at room temperature (22° C.). For this, the microtip electrodes are fixed in parallel at a distance of 27.0 mm on the millimeter gauge and connected to the resistance meter. The electrical resistance is measured directly on the hydrous antiperspirant sticks. For this, the usually domed surface of the antiperspirant sticks is removed using a knife until there is a plane cutting surface. Immediately afterwards, the measurement electrodes are inserted perpendicularly about 5 mm into the stick mass. The measurement value of the electrical resistance is read off after 30 seconds. The measurement electrodes are cleaned using an alcohol-impregnated cellulose cloth. Under the measurement conditions specified, tap water has an electrical resistance of 250 kΩ, a 20% strength by weight aqueous aluminum chlorhydrate solution 3 kΩ and completely demineralized water 1.7 MΩ.

Particularly preferred deodorant or antiperspirant sticks according to the invention are characterized in that pigments, e.g., titanium dioxide, are also present. The pigment content enhances the cosmetic acceptance of the preparation for the user. In addition, particularly preferred deodorant or antiperspirant sticks according to the invention are characterized in that they comprise the customary constituents of cosmetic preparations, e.g., dyes, nanospheres, preservatives and light stabilizers, antioxidants, enzymes and care substances. These are present in particularly preferred deodorant or antiperspirant sticks according to the invention, preferably in an amount of 0.001-20% by weight.

Particularly preferred deodorant or antiperspirant sticks according to the invention are characterized in that, for product stabilization, they comprise at least one free-radical scavenger substance, particularly preferably a substance with the INCI name tris(tetramethylhydroxypiperidinol)citrate, which is available, for example, under the trade name Tinogard Q from Ciba. Tris(tetramethylhydroxypiperidinol) citrate is preferably present in amounts of 0.01-0.1% by weight, particularly preferably, 0.025-0.05% by weight, based on the total weight of the composition according to the invention.

Other particularly preferred deodorant or antiperspirant sticks according to the invention are characterized in that they comprise at least one UV filter. Here, the UV filters are preferably chosen from benzotriazole derivatives, in particular 2-(5-chloro-2H-benzotriazol-2-yl)-6-(1,1-dimethylethyl)-4-methyl-phenol (INCI name: Bumetrizole, obtainable, for example, under the trade name Tinogard AS from Ciba), 2,2′-methylenebis(6-(2H-benzotriazol-2-yl)-4-(1,1,3,3-tetramethylbutyl)phenol) [Tinosorb M (Ciba)], 2,2′-methylbis[6(2H-benzotriazol-2-yl)-4-(methyl)phenol] (MIXXIM BB/200 from Fairmount Chemical), 2-(2′-hydroxy-3′,5′-di-t-amylphenyl)benzotriazole (CAS No.: 025973-551), 2-(2′-hydroxy-5′-octylphenyl)benzotriazole (CAS No. 003147-75-9), 2-(2′-hydroxy-5′-methylphenyl)benzotriazole (CAS No. 2440-22-4), 2-(2H-benzotriazol-2-yl)-4-methyl-6-[2-methyl-3-[1,3,3,3-tetramethyl-1-((trimethylsilyl)oxy]disiloxanyl)propyl]phenol (CAS No.: 155633-54-8) with the INCI name Drometrizole Trisiloxane, 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine or else Aniso Triazine, obtainable as Tinosorb® S from CIBA), 2,4-bis{[4-(3-sulfonato)-2-hydroxypropyloxy)-2-hydroxy]-phenyl)-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl)-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis([4-tris(trimethylsiloxysilylpropyloxy)-2-hydroxy]phenyl}-6-(4-methoxyphenyl)-1,3,5-triazine, 2,4-bis-{([4-(2-methylpropenyloxy)-2-hydroxy]phenyl}-6-(4-methoxyphenyl)-1,3,5-triazine, 2,4-bis([4-(1′,1′,1′,3′,5′,5′,5′-heptamethylsiloxy-2-methyl-propyloxy)-2-hydroxy]phenyl}-6-(4-methoxyphenyl)-1,3,5-triazine and mixtures of said components. Furthermore, the addition of water-soluble UV filters is preferred. Preferred water-soluble UV filters are 2-phenylbenzimidazole-5-sulfonic acid, phenylene-1,4-bis(2-benzimidazyl)-3,3′-5,5′-tetrasulfonic acid and the alkali metal, alkaline earth metal, ammonium, alkylammonium, alkanolammonium and glucammonium salts thereof, in particular, the sulfonic acid itself with the INCI name Phenylbenzimidazole Sulfonic Acid (CAS No. 27503-81-7), which is obtainable, for example, under the trade name Eusolex 232 from Merck or under Neo Heliopan Hydro from Symrise, and the phenylene-1,4-bis(2-benzimidazyl)-3,3′-5,5′-tetrasulfonic acid bis-sodium salt with the INCI name Disodium Phenyl Dibenzimidazole Tetrasulfonate (CAS No.: 180898-37-7), which is available, for example, under the trade name Neo Heliopan AP from Symrise, sulfonic acid derivatives of benzophenones, preferably, 2-hydroxy-4-methoxybenzophenone-5-sulfonic acid and its salts and sulfonic acid derivatives of 3-benzylidenecamphor, such as, for example, 4-(2-oxo-3-bornylidenemethyl)benzenesulfonic acid.

Further particularly preferred deodorant or antiperspirant sticks according to the invention are characterized in that, for product stabilization, they comprise the free-radical scavenger tris(tetramethylhydroxypiperidinol) citrate and the UV filter bumetrizole. Bumetrizole is present preferably in amounts of 0.01-0.1% by weight, particularly preferably, 0.025-0.05% by weight, based on the total weight of the composition according to the invention.

Further particularly preferred deodorant or antiperspirant sticks according to the invention are characterized in that, for product stabilization, they comprise at least one complexing substance. The complexing substances are particularly preferably ethylenediaminetetraacetic acid (EDTA) and its sodium salts, as is available, for example, under the trade name Triton B from BASF, also nitrilotriacetic acid (NTA) and its sodium salts, β-alaninediacetic acid and its salts and phosphonic acids and salts thereof. The at least one complexing substance is present preferably in a total amount of from 0.01-0.5% by weight, particularly preferably 0.08-0.2% by weight, based on the total weight of the composition according to the invention.

Further extraordinarily preferred deodorant or antiperspirant sticks according to the invention are characterized in that they comprise at least one free-radical scavenger substance and at least one substance chosen from UV filters and complexing substances.

Further extraordinarily preferred deodorant or antiperspirant sticks according to the invention are characterized in that they comprise at least one free-radical scavenger substance, at least one UV filter and at least one complexing substance.

Further extraordinarily preferred deodorant or antiperspirant sticks according to the invention are characterized in that they comprise tris(tetramethylhydroxypiperidinol) citrate, bumetrizole and ethylenediaminetetraacetic acid, the latter optionally in the form of the sodium salt.

Further particularly preferred deodorant or antiperspirant sticks according to the invention are characterized in that they comprise at least one hair growth-inhibiting substance. Suitable substances which inhibit hair growth are chosen, in particular, from eflornithine, active ingredient combinations of soya protein hydrolysate, urea, menthol, salicylic acid and extracts of Hypericum perforatum, Hamamelis virginiana, Arnica montana and the bark of Salix alba, as is present, for example and preferably, in the raw material Pilinhib® Veg LS 9109 from Laboratoires Sérobiologiques with the INCI declaration “Propylene glycol, Hydrolyzed Soy Protein, Hypericum Perforatum Extract, Hamamelis Virginiana Extract, Arnica Montana Flower Extract, Urea, Salix Alba Bark Extract, Menthol, Salicylic acid,” also active ingredient combinations of extracts of Epilobium angustifolium, the seeds of Cucurbita pepo (pumpkin) and the fruits of Serenoa serrulata, as are present, for example and preferably in the raw material ARP 100 from Greentech S.A./Rahn with the INCI declaration “Water, Alcohol, Serenoa, Serrulata Fruit Extract, Epilobium Angustifolium Extract, Cucurbita Pepo (Pumpkin) Seed Extract,” also active ingredient combinations of xylitol and extracts of Citrus medica limonum (lemon) fruit, Carica papaya (papaya) and olive leaves, as are present, for example, and preferably, in the raw material xyleine from lmpag/Seporga with the INCI declaration “Xylitol and Citrus Medica Limonum (Lemon) Fruit Extract and Carica Papaya (Papaya) Fruit Extract and Olea europaea (olive) leaf extract,” also active ingredient combinations of Humulus lupulus, Viscum album, Salvia officinalis, Carica papaya and Thuya occidentalis, as are present, for example, and preferably, in the raw material Plantafluid Komplex AH from Plantapharm with the INCI declaration “Aqua, Propylene Glycol, Humulus Lupulus, Viscum Album, Salvia Officinalis, Carica Papaya, Thuya Occidentalis,” and extracts of Larrea divaricata, as are present, for example and preferably in the raw material Capislow from Sederma, the lecithin vesicle with a hydroglycolic extract of Larrea divaricata.

The compositions according to the invention comprise at least one hair growth-inhibiting substance preferably in an amount of from 0.1-10% by weight, preferably 0.5-5% by weight and particularly preferably 1-4% by weight, in each case based on the weight of the raw material tel quel and the total weight of the composition according to the invention.

The customary preservatives can also be added to the compositions according to the invention in order to prevent spoilage of the product as a result of microbial growth. Numerous preservatives also automatically have deodorizing properties, meaning that some substances belong to both groups. Suitable preservatives preferred for cosmetics are, for example, benzoic acid and derivatives thereof (e.g., propyl, phenyl and butyl benzoate, ammonium, sodium, potassium and magnesium benzoate), propionic acid and derivatives thereof (e.g., ammonium, sodium, potassium and magnesium propionate), salicylic acid and derivatives thereof (e.g., sodium, potassium and magnesium salicylate), 4-hydroxybenzoic acid and esters thereof and alkali metal salts (e.g., methyl-, ethyl-, propyl-, isopropyl-, butyl-, isobutyl-, isodecyl-, phenyl-, phenoxyethyl- and benzylparaben, hexamidineparaben and -diparaben, sodium and potassium paraben, sodium and potassium methylparaben, potassium butylparaben, sodium and potassium propylparaben), alcohols and salts thereof (e.g., ethanol, propanol, isopropanol, benzyl alcohol, phenethyl alcohol, phenol, potassium phenoxide, phenoxyethanol, phenoxyisopropanol, o-phenylphenol), guaiacol and derivatives thereof, chlorhexidine and derivatives thereof (e.g., chlorhexidine diacetate, -digluconate, and -dihydrochloride), hydantoin and derivatives thereof (e.g., DEDM- and DMDM-hydantoin, DEDM-hydantoin dilaurate), urea and urea derivatives (e.g., diazolidinylurea, imidazolidinylurea), ferulic acid and derivatives thereof (e.g., ethyl ferulate), sorbic acid and derivatives thereof (e.g., isopropyl sorbate, TEA sorbate, sodium, potassium and magnesium sorbate), isothiazol and oxazole derivatives (e.g., methylisothiazolinone, methylchloroisothiazolinone, dimethyloxazolidine), quaternary ammonium compounds (e.g., polyquatemium-42, quatemium-8, quatemium-14, quatemium-15), carbamates (e.g. iodopropynyl butyl carbamate), formaldehyde and sodium formate, glutaraldehyde, glyoxal, hexamidine, dehydracetic acid, 2-bromo-2-nitropropane-1,3-diol, isopropylcresol, methyldibromoglutaronitrile, polyaminopropylbiguanide, sodium hydroxymethylglycinate, sodium phenolsulfonate, triclocarban, triclosan, zinc pyrithione and various peptide antibiotics (e.g., Nisin).

Preservatives preferred according to the invention are phenoxyethanol, the esters of 4-hydroxybenzoic acid, in particular, methyl-, ethyl-, propyl-, isopropyl-, butyl- and isobutylparaben, and iodopropynyl butylcarbamate.

The amount of preservative in the preferred compositions according to the invention is 0.001-10% by weight, preferably 0.01-5% by weight and in particular, 0.1-3% by weight, based on the total weight of the composition.

In principle, the subject matter of the present invention is also to be broadened to other cosmetic stick compositions which are not deodorant or antiperspirant sticks. A content of deodorant or antiperspirant active ingredients is not obligatory in such sticks. Corresponding sticks can, for example, be formulated as lipstick or as concealing stick and be used by topical application to the skin.

The present invention further provides a cosmetic nontherapeutic method of reducing body odor which is characterized in that a cosmetic deodorant and/or antiperspirant composition is applied to the skin, in particular, to the skin of the axillae.

The present invention further provides a process for the preparation of a deodorant or antiperspirant stick, where the wax and oil components are heated together with the oil-in-water emulsifier(s) and the water-in-oil emulsifier(s) to 90-95° C. and melted, then the water having the water-soluble (active) ingredients and likewise heated to 90-95° C. is added with vigorous stirring, optionally further ingredients are mixed in, the mixture is cooled to a suitable pour temperature, poured into suitable dispenser molds and solidified by static cooling (without further stirring) to room temperature.

The examples below aim to illustrate the subject matter of the invention without limiting it thereto.

TABLE 1
Example compositions according to the invention:

| | Example No. | | | | | | |
|---|---|---|---|---|---|---|---|
| | 1 | 2 | 3 | 4 | 5 | 6 | 7 |
| Lorol ® C18 | — | — | — | — | 2 | — | — |
| Cutina ® MD | 2.4 | 2.4 | 2.4 | 2.4 | — | 2.4 | 2.4 |
| Cutina ® FS45 | 3.3 | 2.5 | 2.5 | 2.5 | 3.3 | 2.5 | 2.5 |
| Eumulgin ® B2 | 1 | 1 | 1 | 1 | 1 | 1 | 1 |
| Eumulgin ® B3 | 1 | 1 | 1 | 1 | 1 | 1 | 1 |
| Eutanol ® G | — | — | — | 6 | — | — | — |
| Diisopropyl adipate | — | 8 | — | — | — | 6 | — |
| Cetiol ® B | — | — | — | — | — | — | 6 |
| Myritol ® 318 | 8 | — | — | — | 8 | — | — |
| Myritol ® 331 | — | — | 8 | — | — | — | — |
| Novata ® AB | 4 | 4 | 4 | 6 | 4 | 6 | 4 |
| Cutina ® CP | 5 | 5 | 5 | 5 | 5 | 5 | 5 |
| Cutina ® HR | 4 | 4 | 4 | 4 | 4 | 4 | 4 |
| Kesterwachs K80 H | 4 | 4 | 4 | 4 | 4 | 4 | 4 |
| Locron ® L | 40 | 40 | 40 | 40 | 40 | 40 | 40 |
| (ACH solution 50% | | | | | | | |
| strength) | | | | | | | |
| Talkum Pharma G | 8 | 8 | 12 | 10 | 8 | 10 | 10 |
| Vitacel 600/20 FCC | — | — | — | — | — | — | 1 |
| Perfume | 1.2 | 1.2 | 1.2 | 1.2 | 1.2 | 1.2 | 1.2 |
| 1,3-Butanediol | — | — | — | — | — | — | — |
| 1,2-Propanediol | 15 | 15 | 12 | 12 | 15 | 12 | 12 |
| Water, demineralized | 3.1 | 3.9 | 2.9 | 4.9 | 3.5 | 4.9 | 5.9 |
| Total | 100.0 | 100.0 | 100.0 | 100.0 | 100.0 | 100.0 | 100.0 |
| Penetration g-force [g]/ | 425 | 380 | 396 | 395 | 425 | 353 | 336 |
| TA15 cone 45°/ | | | | | | | |
| 5 mm/2 mm/s (average | | | | | | | |
| value) | | | | | | | |
| Electrical resistance | 57 | 39 | 57 | 38 | 47 | 39 | 43 |
| 27 mm [kilo-ohm, k′Ω] | | | | | | | |
| Tactile firmness of the | firm | firm | firm | firm | firm | firm | firm |
| mass (sensorially) | | | | | | | |
| Solubility parameter oil | 8.29 | 8.46 | 8.2-8.4 | 8.92 | 8.29 | 8.46 | 8.3-8.6 |
| [(cal/cm³)^{0.5}] | | | | | | | |
| Solubility parameter | 8.28 | 8.28 | 8.28 | 8.28 | 8.9 | 8.28 | 8.28 |
| W/O emulsifier | | | | | | | |
| [(cal/cm³)^{0.5}] | | | | | | | |
| Difference in solubility | 0.01 | 0.18 | −0.08-0.12 | 0.64 | −0.61 | 0.18 | 0.02-0.32 |
| parameters [(cal/cm³)^{0.5}] | | | | | | | |
| Lipid > 50° C. | 11.3 | 10.5 | 10.5 | 10.5 | 11.3 | 10.5 | 10.5 |
| O/W emulsifier | 2 | 2 | 2 | 2 | 2 | 2 | 2 |
| W/O emulsifier | 2.4 | 2.4 | 2.4 | 2.4 | 2 | 2.4 | 2.4 |
| Oil(s) | 8 | 8 | 8 | 6 | 8 | 6 | 6 |
| Lipid 25-<50° C. | 9 | 9 | 9 | 11 | 9 | 11 | 9 |
| Water | 23.1 | 23.9 | 22.9 | 24.9 | 23.5 | 24.9 | 25.9 |

All amounts are given in % by weight.

Example compositions according to the invention and comparison
examples

| | Example No. | | | |
|---|---|---|---|---|
| | 8 | 9 | 10 | 11 |
| Component | according to the | comparison | comparison example | according to the |
| | invention | example | | invention |
| Lorol ® C18 | 4 | — | — | — |
| Cutina ® MD | 3 | 2.4 | 2.4 | 2.4 |
| Cutina ® FS45 | 4 | 3.3 | 3.3 | 3.3 |
| Eumulgin ® B1 | 0.5 | 0.4 | 0.4 | — |
| Eumulgin ® B2 | 1 | 0.8 | 0.8 | 1 |
| Eumulgin ® B3 | 1 | 0.8 | 0.8 | 1 |
| Promyristyl PM 3 | — | — | — | 8 |
| Eutanol ® G16 | 5 | — | — | — |
| Silicone oil DC 245 | — | 8 | — | — |
| Baysilon M350 | 2 | — | — | — |
| Nexbase 2004 FG | — | — | 8 | — |
| Cetiol ® SB 45 | 1 | — | — | — |
| Novata ® AB | — | 4 | 4 | 4 |
| Cutina ® CP | 6 | 5 | 5 | 5 |
| Cutina ® HR | 5 | 4 | 4 | 4 |
| Kesterwachs K80 H | 5 | 4 | 4 | 4 |
| Locron L | 40 | 40 | 40 | 40 |
| (ACH solution 50% | | | | |
| strength) | | | | |
| Talkum Pharma G | — | 8 | 8 | 8 |
| Perfume | 1.2 | 1.2 | 1.2 | 1.2 |
| 1,3-Butanediol | 4 | — | — | — |
| 1,2-Propanediol | — | 15 | 15 | 15 |
| Water, demineralized | 17.3 | 3.1 | 3.1 | 3.1 |
| Total | 100.0 | 100.0 | 100.0 | 100.0 |
| Penetration g-force [g]/ | 171 | 93 | 45 | 407 |
| TA15 cone 45°/5 mm/2 mm/s | | | | |
| (average value) | | | | |
| Electrical resistance | 19 | 55 | 49 | 56 |
| 27 mm [k′Ω] | | | | |
| Tactile firmness of the | plasti-cally firm | soft-elastic/ | very soft, creamy | firm |
| mass (sensorially) | | plastic | | |
| Solubility parameter oil | 8.04 | 5.77 | <7.6 | 8.2-8.4 |
| [(cal/cm³)^{0.5}] | | | | |
| Solubility parameter W/O | 8.63 | 8.28 | 8.28 | 8.28 |
| emulsifier [(cal/cm³)^{0.5}] | | | | |
| Difference in solubility | −0.57 | −2.51 | at least | −0.08-0.12 |
| parameters [(cal/cm³)^{0.5}] | | | −0.68 | |
| Lipid > 50° C. | 14 | 11.3 | 11.3 | 11.3 |
| O/W emulsifier | 2.5 | 2 | 2 | 2 |
| W/O emulsifier | 7 | 2.4 | 2.4 | 2.4 |
| Oil(s) | 7 | 8 | 8 | 8 |
| Lipid 25-<50° C. | 7 | 9 | 9 | 9 |
| Water | 37.3 | 23.1 | 23.1 | 23.1 |

Further example compositions according to the invention:

| | Example No. | | | | | | | | |
|---|---|---|---|---|---|---|---|---|---|
| Component | 12 | 13 | 14 | 15 | 16 | 17 | 18 | 19 | 20 |
| Lorol ® C18 | — | — | — | — | 2 | 2 | — | — | — |
| Cutina ® MD | 2.4 | 2.4 | 2.4 | 2.4 | — | — | 2.4 | 2.4 | 2.4 |
| Cutina ® FS45 | 3.3 | 2.5 | 2.5 | 2.5 | 3.3 | 3.5 | 3.5 | 3.5 | 3.5 |
| Eumulgin ® B2 | 1 | 1 | 1 | 1 | 1 | 0.8 | 0.8 | 0.8 | 1 |
| Eumulgin ® B3 | 1 | 1 | 1 | 1 | 1 | 0.8 | 0.8 | 0.8 | 1 |
| Eutanol ® G | — | — | — | 6 | — | — | — | — | — |
| Diisopropyl adipate | — | 8 | — | — | — | 6 | 6 | 6 | 6 |
| Myritol ® 318 | 8 | — | — | — | 8 | — | — | — | — |
| Myritol ® 331 | — | — | 8 | — | — | — | — | — | — |
| Novata ® AB | 4 | 4 | 4 | 6 | 4 | 4 | 4 | 4 | 4 |
| Cutina ® CP | 5 | 5 | 5 | 5 | 5 | 5 | 5 | 5 | 5 |
| Cutina ® HR | 4 | 4 | 4 | 4 | 4 | 4 | 4 | 4 | 4 |
| Kesterwachs K80 H | 4 | 4 | 4 | 4 | 4 | — | — | — | — |
| Kesterwachs K62 | — | — | — | — | — | 5 | 5 | 5 | 5 |
| Aluminum Zirconium | — | — | 40 | — | — | — | — | — | — |
| Tetrachlorohydrex Gly, 35% | | | | | | | | | |
| solution (Rezal 36 G solution) | | | | | | | | | |
| Aluminum Zirconium | — | — | — | 40 | 40 | — | — | — | — |
| Pentachlorhydrate 40% | | | | | | | | | |
| solution | | | | | | | | | |
| Locron L | — | — | — | — | — | 40 | 40 | 40 | 40 |
| Talkum Pharma G | 8 | 8 | 12 | 10 | 8 | 10 | 10 | 8 | 8 |
| Perfume | 1.2 | 1.2 | 1.2 | 1.2 | 1.2 | 1.2 | 1.2 | 1.2 | 1.2 |
| Tinogard Q | 0.03 | — | 0.05 | — | — | — | 0.05 | 0.05 | — |
| Tinogard HS | 0.2 | — | 0.4 | — | — | — | — | — | — |
| Tinogard AS | — | — | — | — | — | — | — | 0.025 | — |
| Trilon B liquid | — | — | — | — | — | 0.2 | — | — | 0.2 |
| 1,2-Propanediol | 15 | 15 | 12 | 12 | 15 | 12 | 12 | 12 | 15 |
| Water, demineralized | ad 100 | ad 100 | ad 100 | ad 100 | ad 100 | ad 100 | ad 100 | ad 100 | ad 100 |
| Tactile firmness of the mass | firm | firm | firm | firm | firm | firm | firm | firm | firm |
| (sensorially) | | | | | | | | | |
| Solubility parameter oil | 8.29 | 8.46 | 8.46 | 8.46 | 8.46 | 8.46 | 8.46 | 8.46 | 8.46 |
| [(cal/cm³)^{0.5}] | | | | | | | | | |
| Solubility parameter W/O | 8.28 | 8.28 | 8.28 | 8.28 | 8.9 | 8.9 | 8.28 | 8.28 | 8.28 |
| emulsifier [(cal/cm³)^{0.5}] | | | | | | | | | |
| Difference in solubility | 0.01 | 0.18 | 0.18 | 0.18 | −0.44 | −0.44 | 0.18 | 0.18 | 0.18 |
| parameters [(cal/cm³)^{0.5}] | | | | | | | | | |
| Lipid > 50° C. | 11.3 | 10.5 | 10.5 | 10.5 | 11.3 | 12.5 | 12.5 | 12.5 | 12.5 |
| (% by wt.) | | | | | | | | | |
| O/W emulsifier (% by wt.) | 2 | 2 | 2 | 2 | 2 | 1.6 | 1.6 | 1.6 | 2 |
| W/O emulsifier (% by wt.) | 2.4 | 2.4 | 2.4 | 2.4 | 2 | 2 | 2.4 | 2.4 | 2.4 |
| Oil(s) (% by wt.) | 8 | 8 | 8 | 6 | 8 | 6 | 6 | 6 | 6 |
| Lipid 25-<50° C. (% by wt.) | 9 | 9 | 9 | 11 | 9 | 9 | 9 | 9 | 9 |
| Water (% by wt.) | 22.87 | 23.9 | 28.45 | 28.9 | 27.5 | 26.3 | 25.25 | 27.225 | 23.7 |

List of raw materials used:

| | | Supplier/ |
|---|---|---|
| Component | INCI | Manufacturer |
| Baysilone ® oil M 350 | Dimethicone | GE Bayer Silicones |
| Cetiol ® B | Dibutyl Adipate | Cognis |
| Cetiol ® SB 45 | Butyrospermum Parkii (Shea Butter) | Cognis |
| Cutina ® CP | Cetyl Palmitate | Cognis |
| Cutina ® FS45 | Palmitic Acid, Stearic Acid | Cognis |
| Cutina ® HR | Hydrogenated Castor Oil | Cognis |
| Cutina ® MD | Glyceryl Stearate | Cognis |
| Eumulgin ® B1 | Ceteareth-12 | Cognis |
| Eumulgin ® B2 | Ceteareth-20 | Cognis |
| Eumulgin ® B3 | Ceteareth-30 | Cognis |
| Eutanol ® G | Octyldodecanol | Cognis |
| Eutanol ® G 16 | Hexyldecanol | Cognis |
| Kesterwachs K 62 | Cetearylbehenat | Koster Keunen |
| Kesterwachs K80 H | C20-40 Alkyl Stearate | Koster Keunen |
| Locron L | Aluminum Chlorhydrate | Clariant |
| (ACH solution 50% strength) | | |
| Lorol ® C18 | Stearyl Alcohol | Cognis |
| Myritol ® 318 | Caprylic/Capric Triglyceride | Cognis |
| Myritol ® 331 | Cocoglycerides | Cognis |
| Nexbase 2004 FG | Hydrogenated Polydecene | Fortum |
| Novata ® AB | Cocoglycerides | Cognis |
| Promyristyl ® PM 3 | PPG-3 Myristyl Ether | Croda |
| Silicone oil DC 245 | Cyclopentasiloxane | Dow Corning |
| Talkum Pharma G | Talc | |
| Tinogard AS | Bumetrizole | Ciba |
| Tinogard HS | Sodium Benzotriazolyl Butylphenol Sulfonate | Ciba |
| Tinogard Q | Tris(tetramethylhydroxypiperidinol)citrate | Ciba |
| Trilon B liquid | Water, TETRASODIUM EDTA (39-41% by wt. active | BASF |
| | substance) | |

1-20. (canceled)

21. Deodorant or antiperspirant stick comprising:
a) at least one lipid or wax component with a melting point of >50° C., wherein the at least one lipid or wax component a) is not a component b) or c),
b) at least one nonionic oil-in-water emulsifier with an HLB value of more than 7 within an oil-in-water emulsifier mixture with an average HLB value in the range from 10-19,
c) at least one nonionic water-in-oil emulsifier with an HLB value greater than 1.0 and less than or equal to 7.0, which can form liquid crystal structures with water on its own or with water in the presence of a hydrophilic emulsifier, as consistency regulator and/or water binder,
d) at least one oil liquid at 20° C., which is not a fragrance component or an essential oil, e) at least one water-soluble polyhydric C₂-C₉-alkanol with 2-6 hydroxyl groups and/or at least one water-soluble polyethylene glycol with 3-20 ethylene oxide units,
f) 5 to less than 50% by weight of water, based on the overall composition,
g) at least one deodorant and/or antiperspirant active ingredient,
wherein the (average) solubility parameter of the oils d) in the presence of linear saturated fatty alcohols c) having a chain length of at least 8 carbon atoms deviates by at most −0.7 (cal/cm³)^{0.5} or at most +0.7 (cal/cm³)^{0.5}, and in the presence of water-in-oil emulsifiers c) different from linear saturated fatty alcohols with a chain length of at least 8 carbon atoms deviates by at most −0.4 (cal/cm³)^{0.5} or at most +0.7 (cal/cm³)^{0.5} from the (average) solubility parameter of water-in-oil emulsifiers, and
wherein the stick is in the form of an oil-in-water dispersion and has
i) a penetration force value of from 150-800 gram-force (g-force) at a penetration depth of 5000 mm and
ii) an electrical resistance of at most 300 kΩ (kiloohm).

22. Deodorant or antiperspirant stick according to claim 21 wherein the lipid or wax component a) is present in an amount of 4-20% by weight, based on total weight of the composition.

23. Deodorant or antiperspirant stick according to claim 21, wherein the oil-in-water emulsifier b) is present in an amount of 0.5-10% by weight, based on total weight of the composition.

24. Deodorant or antiperspirant stick according to claim 21, wherein the water-in-oil emulsifier c) is present in an amount of 0.1-15% by weight, based on total weight of the composition.

25. Deodorant or antiperspirant stick according to claim 21, wherein the oil d) liquid at 20° C. is present in an amount of 3-20% by weight, based on total weight of the composition.

26. Deodorant or antiperspirant stick according to claim 21, wherein the water-soluble polyhydric C₂-C₉-alkanol with 2-6 hydroxyl groups and/or the water-soluble polyethylene glycol with 3-20 ethylene oxide units e) is present in an amount of 3-25% by weight, based on total weight of the composition.

27. Deodorant or antiperspirant stick according to claim 21, wherein the deodorant active ingredient is present in an amount of 0.1-10% by weight, and/or the antiperspirant active ingredient is present in a total amount of 3-25% by weight, each based on total weight of active substance in the total composition.

28. Deodorant or antiperspirant stick as claimed in claim 21, wherein the lipid or wax component a) is chosen from esters of saturated, monohydric C₁₆-C₆₀ alkanol and saturated C₈-C₃₆ monocarboxylic acid, glycerol triesters of saturated linear C₁₂-C₃₀ carboxylic acids which may be hydroxylated, candelilla wax, carnauba wax, beeswax, saturated linear C₁₄-C₃₆ carboxylic acids, and mixtures thereof.

29. Deodorant or antiperspirant stick according to claim 28 wherein the ester/esters of saturated, monohydric C₁₆-C₆₀-alkanol and saturated C₈-C₃₆-monocarboxylic acid are present in an amount of 2-10% by weight, based on total weight of the composition.

30. Deodorant or antiperspirant stick according to claim 21 wherein the nonionic oil-in-water emulsifier b) with an HLB value of more than 7 is chosen from ethoxylated C₈-C₂₄-alkanols with, on average, 10-100 mol of ethylene oxide per mole; ethoxylated C₈-C₂₄-carboxylic acids with an average of 10-100 mol of ethylene oxide per mole; silicone copolyols with ethylene oxide units or with ethylene oxide and propylene oxide units; alkyl mono- and oligoglycosides having 8 to 22 carbon atoms in the alkyl radical and ethoxylated analogs thereof; ethoxylated sterols; partial esters of polyglycerols with n =2 to 10 glycerol units and esterified with 1 to 4 saturated or unsaturated, linear or branched, optionally hydroxylated C₈-C₃₀-fatty acid radicals, having an HLB value of more than 7; and mixtures thereof.

31. Deodorant or antiperspirant stick according to claim 21 wherein the nonionic water-in-oil emulsifier c) is chosen from linear saturated C₁₂-C₃₀-alkanols; ethylene glycol mono- and diesters of linear, saturated and unsaturated C₁₂-C₃₀-carboxylic acids, which may be hydroxylated; glycerol mono- and diesters of linear, saturated and unsaturated C₁₂-C₃₀-carboxylic acids, which may be hydroxylated; propylene glycol mono- and diesters of linear, saturated and unsaturated C₁₂-C₃₀-carboxylic acids, which may be hydroxylated; sorbitan mono-, di- and triesters of linear, saturated and unsaturated C₁₂-C₃₀-carboxylic acids, which may be hydroxylated; pentaerythrityl mono-, di-, tri- and tetraesters of linear, saturated and unsaturated C₁₂-C₃₀-carboxylic acids, which may be hydroxylated; methylglucose mono- and diesters of linear, saturated and unsaturated C₁₂-C₃₀-carboxylic acids, which may be hydroxylated; sterols; alkanols and carboxylic acids having 8-24 carbon atoms in the alkyl group and 1-4 ethylene oxide units per molecule and an HLB value of greater than 1.0 and less than or equal to 7.0; glycerol monoethers of saturated and/or unsaturated, branched and/or unbranched alcohols of chain length from 8-30 carbon atoms; partial esters of polyglycerols having 2 to 10 glycerol units and esterified with 1 to 5 saturated or unsaturated, linear or branched, optionally hydroxylated C₈-C₃₀-fatty acid radicals having an HLB value of less than or equal to 7; and mixtures thereof

32. Deodorant or antiperspirant stick according to claim 21, wherein the oil d) liquid at 20° C. is chosen from branched saturated or unsaturated fatty alcohols having 6-30 carbon atoms; triglycerides of linear or branched, saturated or unsaturated, optionally hydroxylated C₈₋₃₀-fatty acids; dicarboxylic acid esters of linear or branched C₂-C₁₀-alkanols; esters of branched saturated or unsaturated fatty alcohols having 2-30 carbon atoms with linear or branched saturated or unsaturated fatty acids having 2-30 carbon atoms which may be hydroxylated; addition products of from 1 to 5 propylene oxide units onto mono- or polyhydric C₈₋₂₂-alkanols; addition products of at least 6 ethylene oxide and/or propylene oxide units onto mono- or polyhydric C₃₋₂₂-alkanols; C₈-C₂₂-fatty alcohol esters of monobasic or polybasic C₂-C₇-hydroxycarboxylic acids; symmetrical, asymmetrical or cyclic esters of carbonic acid with fatty alcohols; esters of dimers of unsaturated C₁₂-C₂₂-fatty acids (dimer fatty acids) with monohydric linear, branched or cyclic C₂-C₁₈-alkanols or with polyhydric linear or branched C₂-C₆-alkanols; and mixtures thereof.

33. Deodorant or antiperspirant stick according to claim 21, wherein the oils d) liquid at 20° C. comprise at most 20% by weight of oil(s), based on total weight of oils liquid at 20° C., whose solubility parameter deviates by more than −0.4 or −0.7 (cal/cm³)^{0.5} or by more than +0.7 (cal/cm³)^{0.5} from the (average) solubility parameter of the at least one water-in-oil emulsifier c).

34. Deodorant or antiperspirant stick according to claim 21, wherein no oils liquid at 20° C. are present whose solubility parameter deviates by more than ±1.0 (cal/cm³)^{0.5} from the (average) solubility parameter of the at least one water-in-oil emulsifier c).

35. Deodorant or antiperspirant stick according to claim 1, wherein the at least one water-soluble polyhydric C₂-C₉-alkanol e) with 2-6 hydroxyl groups is chosen from 1,2-propylene glycol, 2-methyl-1,3-propanediol, glycerol, butylene glycols, such as 1,2-butylene glycol, 1,3-butylene glycol and 1,4-butylene glycol, pentylene glycols, such as 1,2-pentanediol and 1,5-pentanediol, hexanediols, such as 1,6-hexanediol, hexanetriols, such as 1,2,6-hexanetriol, 1,2-octanediol, 1,8-octanediol, dipropylene glycol, tripropylene glycol, diglycerol, triglycerol, erythritol, sorbitol, and mixtures of the abovementioned substances.

36. Deodorant or antiperspirant stick according to claim 21, wherein the deodorant active ingredient g) is chosen from
arylsulfatase inhibitors, β-glucuronidase inhibitors, aminoacylase inhibitors, esterase inhibitors, lipase inhibitors and lipoxigenase inhibitors; α-monoalkyl glycerol ethers having a branched or linear saturated or unsaturated, optionally hydroxylated C₆-C₂₂-alkyl radical; phenoxyethanol; antimicrobial perfume oils; deosafe perfume oils; prebiotically effective components; trialkylcitric esters; active ingredients which reduce the number or inhibit the growth of skin microbes involved in odor formation; zinc compounds; organohalogen compounds; quaternary ammonium compounds; odor absorbers; sodium bicarbonate; antibiotics; and mixtures thereof,
and/or the antiperspirant active ingredient g) is chosen from the water-soluble astringent inorganic and organic salts of aluminum, zirconium and zinc and mixtures thereof.

37. Deodorant or antiperspirant stick according to claim 21, further comprising at least one lipid or wax component with a melting point in the range from 25-<50° C., chosen from coconut fatty acid glycerol mono-, di- and triesters, Butyrospermum Parkii (shea butter) and esters of saturated, monohydric C₈-C₁₈-alcohols with saturated C₁₂-C₁₈-monocarboxylic acids, and mixtures thereof.

38. Deodorant or antiperspirant stick according to claim 37, wherein the lipid or wax component with a melting point in the range from 25-<50° C. is present in an amount of from 0.01 to 20% by weight, based on total composition.

39. Deodorant or antiperspirant stick according to claim 21, further comprising at least one solid, water-insoluble particulate filler.

40. Deodorant or antiperspirant stick according to claim 39, wherein the solid, water-insoluble particulate filler is chosen from optionally modified starches and starch derivatives, which are pregelatinized if desired, cellulose and cellulose derivatives, silicon dioxide, silicas, spherical polyalkylsesquioxane particles, silica gels, talc, kaolin, clays, magnesium aluminum silicates, boron nitride, lactoglobulin derivatives, glass powder, polymer powders and mixtures thereof.

41. Deodorant or antiperspirant stick according to claim 39, wherein the solid, water-insoluble particulate filler is present in a total amount of from 0.01 to 20% by weight based on total weight of the composition.

42. Deodorant or antiperspirant stick according to claim 21 further comprising at least one fragrance component.

43. Deodorant or antiperspirant stick according to claim 21 further comprising at least one free-radical scavenger substance.

44. Deodorant or antiperspirant stick according to claim 21 further comprising at least one UV filter.

45. Deodorant or antiperspirant stick according to claim 21 further comprising at least one complexing substance.

46. Deodorant or antiperspirant stick according to claim 21 further comprising at least one hair growth-inhibiting substance.

47. Deodorant or antiperspirant stick according to claim 21, wherein the water is present in an amount of 10 to less than 30% by weight, based on total weight of the overall composition.

48. Deodorant or antiperspirant stick according to claim 21, wherein the stick has a penetration force value of from 200-750 gram-force (g-force) at a penetration depth of 5000 mm.

49. Deodorant or antiperspirant stick according to claim 21, wherein the stick has an electrical resistance of at most 100 kΩ.

50. Cosmetic nontherapeutic method of reducing body odor comprising the step of applying a cosmetic deodorant and/or antiperspirant composition according to claims 21 to the skin.

51. Process for the preparation of a deodorant or antiperspirant stick according to claim 21 comprising the steps of:
heating the wax and oil components together with the oil-in-water emulsifier(s) and the water-in-oil emulsifier(s) to 90-95° C. to melt those components,
adding with vigorous stirring water at 90-95° C. containing the water-soluble (active) ingredients,
optionally mixing in other ingredients,
cooling the mixture to a suitable pour temperature,
pouring the mixture into suitable dispenser molds, and
solidifying the mixture by static cooling (without further stirring) to room temperature.
